(19) United States
(12) Patent Application Publication
(10) Pub. No.: US 2013/0203610 A1
(43) Pub. Date: Aug. 8, 2013

(54) Tools and Method for Nanopores Unzipping-Dependent Nucleic Acid Sequencing

(71) Applicants: Amit Meller, Brookline, MA (US); Alon Singer, Brighton, MA (US)

(72) Inventors: Amit Meller, Brookline, MA (US); Alon Singer, Brighton, MA (US)

(73) Assignee: TRUSTEES OF BOSTON UNIVERSITY, Boston, MA (US)

(21) Appl. No.: 13/638,455

(22) PCT Filed: Mar. 30, 2011
(86) PCT No.: PCT/US2011/030430

§ 371 Date: Apr. 17, 2013

Related U.S. Application Data
(60) Provisional application No. 61/318,872, filed on Mar. 30, 2010.

(51) Int. Cl. C12Q 1/68

(52) U.S. Cl. CPC C12Q 1/6874
USPC 506/6; 506/16

(57) ABSTRACT

Provided herein is a library that comprises a plurality of molecular beacons (MBs), each MB having a detectable label, a detectable label blocker and a modifier group. The library is used in conjunction with nanopore unzipping-dependent sequencing of nucleic acids.

### CROSS REFERENCE TO RELATED APPLICATION

This application claims benefit under 35 U.S.C. §119(e) of the U.S. Provisional Application No. 61/318,872 filed Mar. 30, 2010, the contents of which are incorporated herein by reference in its entirety.

### GOVERNMENT SUPPORT

This invention was made with Government support under contract No. RO1-HG004128 awarded by the National Institutes of Health. The Government has certain rights in the invention.

### BACKGROUND OF INVENTION

Nanopore sequencing is a promising technology being developed as a cheap and fast alternative to the conventional Sanger sequencing method. Nanopore sequencing methods can provide several advantages over the conventional Sanger sequencing method; they permit single molecule analysis, are not enzyme dependent (e.g., polymerase enzyme is not required for chain extension), and require significantly less reagents.

A number of nanopore based DNA sequencing methods have recently been proposed¹⁴ and highlight two major challenges¹⁵: 1) The ability to discriminate among individual nucleotides (nt), e.g., the system must be capable of differentiating among the four bases at the single-molecule level, and 2) the method must enable parallel readout.

In nanopore based DNA sequencing methods, it had been previously difficult to scale down DNA analysis to the single molecule level, mainly due to the relatively small differences between the four nucleotides constituting DNA, and due to the inherent noise in single molecule probing. The approach taken by some to circumvent these problems is to ‘magnify” each of the individual bases of a DNA to distinct entities that produces measurable signals that are significantly greater than the background noise level, thereby increasing the signal-to-noise ratio. This is achieved by an initial preparation step of converting the DNA molecules to be analyzed into longer and periodically structured DNA molecule, named “Design Polymers”^{17,29,30}.

Currently, there are two general approaches used in nanopore based DNA sequencing methods for “detecting” or measuring the individual bases of a DNA: 1) by monitoring a change in the pore conductivity when the DNA enters and passes through the pore, the change in the pore conductivity can be measured directly e.g., using an electrometer; and 2) by optical detection of distinct molecular beacons as they are unzipped by a nanopore that must be small enough to exclude a double-stranded DNA but yet will permit the entry and translocation of a single stranded DNA. In the first approach, bulky groups are attached to the bases of nucleotide to increase and make distinct the electronic blockade signals generated for detection when the double-stranded DNA translocate through the nanopore³². In the second approach, the DNA is initially converted to an expanded, digitized form by systematically substituting each and every base in the DNA sequence with a specific ordered pair of concatenated oligonucleotides^{29,31} (FIG. 1). There is a specific species of oligonucleotide representing each of the different bases, e.g., A, T, U, G, or C. The converted DNA is hybridized with complementary molecular beacons to form a double-stranded DNA. There are distinct species of molecular beacons complementary oligonucleotide representing each of the different bases, e.g., A, T, U, G, or C. These different species of molecular beacons are distinctly labeled for identification purposes, e.g., four different fluorophores for four species of molecular beacons. To detect the sequence of the DNA, nanopores of less than 2 nm are then used to sequentially unzip the beacons from the double-stranded DNA (dsDNA) comprising molecular beacons. With each unzipping event a new fluorophore is un-quenched, giving rise to a series of photon flashes in different colors, which are recorded by a CCD camera (FIG. 2). The unzipping process slows down the translocation of the DNA through the pore in a voltage-dependent manner, to a rate compatible with optical recording.

One limiting factor of DNA sequencing that is dependent on nanopore unzipping of a labeled dsDNA is that the pore of the nanopore has to be small enough to pry open the double-stranded structure, usually less than 2 nm in diameter. Currently, there are two general approaches to prepare nanopores for nucleic acid analysis: (1) Organic nanopores that are prepared from naturally occurring molecules, such as alpha-hemolysin pores. Although organic nanopores are commonly used for DNA analysis, organic nanopores are great for single DNA sequencing and not easily adaptable for high throughput DNA sequencing requiring numerous nanopores at the same time. (2) Synthetic solid-state nanopores that are made by various conventional and non-conventional fabrication techniques. Synthetically fabricated nanopores holds more potential for high throughput DNA sequencing requiring numerous nanopores at the same time.

Another limiting factor of DNA sequencing that is dependent on nanopore unzipping of a labeled dsDNA is that a single nanopore can probe only a single molecule at a time. Development of fast, high throughput, genomic sequencing using nanopore base sequencing methods would entail an array of nanopores and the simultaneous monitoring the nanopores. Although fabrication of nanopores can produces lots of synthetic nanopores, uniform constant quality manufacture of nanopores with very small pore is difficult. Alternative strategies in nanopore based unzipping sequencing methods that permit the use of nanopores with slightly larger pore size are desirable.

### SUMMARY OF THE INVENTION

Embodiments of the present invention are based on the discovery that linking a modifier group to a moiety such as a molecular beacon (MB) used in nanopore unzipping-dependent sequencing of nucleic acids enables the use of a nanopore with a larger pore than the width of a standard double stranded (ds) nucleic acid, which is ˜2.2 nm. For nanopore unzipping-dependent sequencing, a pore size of ˜1.5-2.0 nm allows only a single stranded nucleic acid to translocate through the opening of the pore in an electric field. This essentially forces strand separation of the ds nucleic acid in contact with the nanopore, this process is commonly termed “unzipping”. The problem with this conventional method is that the nanopore size is limited to a pore size smaller than that of the width of the ds nucleic acid. The large scale manufacture of small-size nanopores having uniform pore sizes is difficult. The modifier group linked to the MB adds bulk to the MB and allows adaptation of the conventional method to use nanopores with larger pore size. A ds nucleic acid is formed by the hybridization of a single stranded nucleic acid and multiple MBs that each has bulky modifier groups linked thereon. The presence of the bulky modifier group on the MBs serves to increase the width of the ds nucleic acid at the point of attachment of the bulk group to the MB (see FIG. 9) to a width that is greater than the width of a standard double stranded ds nucleic acid. Larger pores that are greater than 2.0 nm but less than that of the width of the ds nucleic acid at the point of attachment of the bulk group to the MB can be used to unzip the ds nucleic acid comprising bulky group linked MBs in the sequencing process. A larger pore of such configuration is still capable of permitting only the single stranded nucleic acid to translocate through the opening of the pore in an electric field. A larger pore of such configuration achieves this by preventing the MB with a linked bulky group from translocating through the opening of the pore in an electric field since the pore is smaller than the th of the ds nucleic acid at the point of attachment of the bulk group to the MB (D3, see FIG. 9). This results in strand separation of the ds nucleic acid just as strand separation would take place with a standard ds nucleic acid and a nanopore size of ˜1.5-2.0 nm, i.e. without bulk group linked MBs. A standard ds nucleic acid which has no bulky modifier groups linked thereon would have a width of approximately 2.2 nm.

As used herein, and unless stated otherwise, each of the following terms shall have the definition set forth below.

“Nanopore” includes, for example, a structure comprising (a) a first and a second compartment separated by a physical barrier, which barrier has at least one pore with a diameter, for example, of from about 1 to 10 nm, and (b) a means for applying an electric field across the barrier so that a charged molecule such as DNA can pass from the first compartment through the pore to the second compartment. The nanopore ideally further comprises a means for measuring the electronic signature of a molecule passing through its barrier. In one embodiment, the nanopore barrier is synthetic, i.e., made of synthetic material or a synthetically made nanopore. In one embodiment, the nanopore barrier is synthetic occurring in part. In one embodiment, the nanopore barrier is natural, i.e., made of natural material or a naturally existing barrier. In one embodiment, the nanopore barrier is naturally occurring in part. Barriers can include, for example, lipid bilayers having therein α-hemolysin, oligomeric protein channels such as porins, and synthetic peptides and the like. In one embodiment, the nanopore barrier can also include inorganic plates having one or more holes of a suitable size. In some embodiments, the nanopore barrier comprises organic and/or inorganic materials. In some embodiments, the nanopore barrier comprises modification of the organic and/or inorganic materials, or synthetic or naturally occurring materials. Herein “nanopore” and the “pore” in the nanopore barrier are used interchangeably.

As used herein, the term “comprising” means that other elements can also be present in addition to the defined elements presented. The use of “comprising” indicates inclusion rather than limitation.

The term “consisting of” in reference to the libraries, methods, and respective components thereof as described herein, means the exclusion of any element or components not recited in that description of the embodiment.

As used herein the term “consisting essentially of” refers to those elements required for a given embodiment. The term permits the presence of elements that do not materially affect the basic and novel or functional characteristic(s) of that embodiment of the invention.

As used herein, the term “nucleic acid” shall mean any nucleic acid molecule, including, without limitation, DNA, RNA and hybrids or analogues thereof. The nucleic acid bases that form nucleic acid molecules can be the bases A, C, G, T and U, as well as derivatives thereof. Derivatives of these bases are well known in the art. A nucleic acid is a macromolecule composed of chains of monomeric nucleotides. In some embodiments, the nucleic acids are deoxyribonucleic acid (DNA) and ribonucleic acid (RNA). In other embodiments, the nucleic acids are artificial nucleic acids such as peptide nucleic acid (PNA), Morpholino, locked nucleic acid (LNA), glycol nucleic acid (GNA) and threose nucleic acid (TNA). Each of these is distinguished from naturally-occurring DNA or RNA by changes to the backbone of the molecule.

As used herein, the term “oligonucleotide” is a polymeric form of nucleotides of any length. Generally, the number of nucleotide units may range from about 2 to 100, and preferably from about 2 to 30 or 50 to 80. In one embodiment, the oligonucleotides of the MBs described herein are 4-25 nucleotides in length. In the context of the library of MBs and methods described herein, the term “oligonucleotide” refers to a plurality of naturally-occurring, non-naturally-occurring, commonly known or synthetic nucleotides joined together in a specific sequence such as glycol nucleic acid (GNA), locked nucleic acid (LNA), peptide nucleic acid (PNA), threose nucleic acid (TNA), and phosphorodiamidate morpholino oligo (PMO/Morpholino). They can be any length, modified or unmodified at their 3′-ends and/or 5′ ends. In one embodiment, the “oligonucleotide” refers to a DNA or an RNA.

As used herein, the term “a polymer comprising defined sequences representative of A, U, T, C or G” when used in the context of the methods described herein refers to a polymer comprising “block sequences” wherein each block sequence, individually or in combination, represents the nucleotide bases A, U, T, C or G. In one embodiment, the “defined sequences representative of A, U, T, C or G” refers to to a polymer comprising “block sequences” wherein each block sequence, individually or in combination, represents the nucleotide bases A, U, T, C or G.

As used herein, a “block sequence” when used in the context of a polymer comprising defined sequences representative of A, U, T, C or G refers to a short nucleic acid of 4-35 nucleotides of a specific sequence, which individually or in combination with another block sequence, is representative of either A, U, T, C or G. For example, ATTTGGAAT is a block-0 and TTCCGAGGT is another block-1. The combination of blocks 01 is ATTTGGAAT-TTCCGAGGT (SEQ. ID. NO: 1) and it represents the nucleotide base A.

In practicing the embodiments of the inventions described herein, one can use the modifier groups attached to any moiety. An exemplary moiety is a molecular beacon. Other moieties include but are not limited to DNAs, RNAs and peptides. Applications of the embodiments of the invention described herein include but are not limited to protein assays or detection using apatmers. For applications in protein detection, the nanopore may be combined with a moiety for specific protein analysis, e.g., a specific protein-binding moiety. However, for the purpose of illustrating the invention, the moiety described herein is a MB. This illustration should not in any way be construed that the moiety is limited only to MBs.

Accordingly, provided herein is a library of molecular beacons (MBs) for nanopore unzipping-dependent sequencing of nucleic acids, the library comprising a plurity of MBs wherein each MB comprises an oligonucleotide that comprises (1) a detectable label; (2) a detectable label blocker; and (3) a modifier group; wherein the MB is capable of sequence-specific complementary hybridization to a defined sequence that is representative of an A, U, T, C, or G nucleotide in a single-stranded nucleic acid to form a double-stranded (ds) nucleic acid.

In one embodiment, provided herein is a method of unzipping a double-stranded (ds) nucleic acid for nanopore unzipping-dependent sequencing of nucleic acids, the method comprising (a) hybridizing the library of molecular beacons (MBs) described herein to a single stranded nucleic acid to be sequenced, thereby forming a double stranded (ds) nucleic acid with a width of D3, which is formed by the presence of the modifier group on the MB, wherein the single stranded nucleic acid to be sequenced is a polymer comprising defined sequences representative of A, U, T, C or G; (b) contacting the ds nucleic acid formed in step a) with an opening of a nanopore with a width of D1, wherein D3 is greater than D1; and (c) applying an electric potential across the nanopore to unzip the hybridized MBs from the single stranded nucleic acid to be sequenced. The electric field produced by the electric potential across the nanopore cause the ds nucleic acid to translocate from one compartment to the other of the nanopore, through the nanopore. During the translocation process, the MB is stripped off the ds nucleic acid at the entrance of the nanopore because the bulk-group-linked MB is too big (i.e. too wide) to translocate through the pore together with the complementarily hybridized single strand nucleic acid.

In another embodiment, provided herein is a method for determining the nucleotide sequence of a nucleic acid comprising the steps of: (a) hybridizing the library of molecular beacons (MBs) described herein to a single stranded nucleic acid to be sequenced, thereby forming a double stranded (ds) nucleic acid with a width of D3, which is formed by the presence of the modifier group on the MB, wherein the single stranded nucleic acid to be sequenced is a polymer comprising defined sequences representative of A, U, T, C or G; (b) contacting the double-stranded nucleic acid formed in step a) with an opening of a nanopore with a width of D1, wherein D3 is greater than D1; (c) applying an electric potential across the nanopore to unzip the hybridized MBs from the single stranded nucleic acid to be sequenced; and (d) detecting a signal emitted by a detectable label from each MB as the MB separates from the ds nucleic acid at the pore. The electric field produced by the electric potential across the nanopore cause the ds nucleic acid to translocate from one compartment to the other of the nanopore, through the nanopore. During the translocation process, the MB is stripped off the ds nucleic acid at the entrance of the nanopore because the bulk-group-linked MB is too big (i.e. too wide) to translocate through the pore together with the complementarily hybridized single strand nucleic acid.

In one embodiment, the method for determining the nucleotide sequence of a nucleic acid further comprising decoding the sequence of detected signals to the nucleotide base sequence of the nucleic acid being sequenced.

In one embodiment, the oligonucleotide of the MB comprises two affinity arms. In some embodiment, the MB oligonucleotide comprises a 5′ affinity arm and a 3′ affinity arm. The affinity arms are portion of the oligonucleotide that have complementary sequence and can hybridize when the conditions are favorable for hybridization.

In one embodiment, the oligonucleotide of the MB comprises 4-60 nucleotides.

In one embodiment, the oligonucleotide is a polymer. In one embodiment, the polymer comprises 4-60, nucleotides, nucleobases or monomers. In one embodiment, the monomers are nucleotides and analogues thereof, e.g., didanosine, vidarabine, cytarabine, emtricitabine, lamivudine, zalcitabine, abacavir, entecavir, stavudine, telbivudine, zidovudine, idoxuridine and trifluridine. In one embodiment, some of the nucleotides, nucleobases or monomers can be modified for the purpose of conjugating with a detectable label, a detectable label blocker, a modifier group, e.g., a thiol-dT.

In one embodiment, the oligonucleotide of the MB comprises a nucleic acid selected from a group consisting of deoxyribonucleic acid (DNA), ribonucleic acid (RNA), glycol nucleic acid (GNA), locked nucleic acid (LNA), peptide nucleic acid (PNA), threose nucleic acid (TNA), and phosphorodiamidate morpholino oligo (PMO/Morpholino). In one embodiment, the monomer of the oligonucleotide is selected from a group consisting of deoxyribonucleic acid (DNA), ribonucleic acid (RNA), glycol nucleic acid (GNA), peptide nucleic acid (PNA), locked nucleic acid (LNA), threose nucleic acid (TNA) and (PMO/Morpholino). In another embodiment, the oligonucleotide of the MB is a chimeric oligonucleotide, i.e., comprising a mixture or combinations of DNA, RNA, GNA, PNA, LNA, TNA and Morpholino. e.g., (DNA+RNA), (GNA+RNA), (LNA+DNA), (PNA+DNA+RNA) etc.

In one embodiment, the oligonucleotide of the MB comprises a pair of “arms'. In one embodiment, the oligonucleotide of the MB comprises a 5′ arm and a 3′ arm, preferably a 5′ fluorophores arm and a 3′ quencher arm. In this embodiment, the detectable label is the fluorophore found on the 5′ fluorophores arm and the detectable label blocker is the quencher found on the 3′ quencher arm of the MB.

In one embodiment, the detectable label is linked on one end of the oligonucleotide of the MB and is on the same end for all oligonucleotides of the MBs in the library. In one embodiment, the detectable label emits a signal that is detected and/or measured when the detectable label is not inhibited by a blocker.

In one embodiment, the MB of the library is not attached to a solid phase carrier. In one embodiment, the MB of the library is free in solution.

In one embodiment, the detectable label, detectable label blocker and the modifier group on the oligonucleotide of the MBs in the library do not interfere with sequence-specific complementary hybridization of the MBs with the define sequence that is representative of an A, U, T, C, or G nucleotide in a single-stranded nucleic acid.

In one embodiment, the detectable group's signal is detected optically, e.g., by light intensity, color of light emitted, or fluorescence etc.

In one embodiment, the detectable group is a fluorophore and the signal is fluorescence.

In one embodiment, the detectable label blocker is a quencher of the fluorophore.

In one embodiment, the detectable label blocker is also the modifier group. In other words, the detectable label blocker and the modifier group on the MB are the same molecule. In other words, the detectable label blocker on the MB also functions as the modifier group.

In one embodiment, the modifier group on the oligonucleotide of the MB increases the width of a ds nucleic acid thus formed therewith at the point of attachment of the modifier group to the oligonucleotide of the MB to greater than 2.0 nanometers (nm), wherein the ds nucleic acid is formed by hybridization of the MBs to the defined sequence that is representative of A, U, T, C, or G. (see FIG. 9). In one embodiment, the modifier group on the oligonucleotide of the MB increases the width of a ds nucleic acid thus formed therewith at the point of attachment of the modifier group to the oligonucleotide of the MB to greater than 2.2 nm, wherein the ds nucleic acid is formed by hybridization of the MBs to the defined sequence that is representative of A, U, T, C, or G. In one embodiment, the modifier group on the oligonucleotide of the MB increases D2 of a ds nucleic acid thus formed therewith to greater than 2.0 nm (see FIG. 9). In one embodiment, the modifier group on the oligonucleotide of the MB increases D2 of a ds nucleic acid thus formed therewith to greater than 2.2 nm (see FIG. 9).

In one embodiment, the modifier group on the oligonucleotide of the MB increases the width of a ds nucleic acid thus formed therewith to greater than 2.0 nm. In one embodiment, the modifier group on the oligonucleotide of the MB increases the width of a ds nucleic acid thus formed therewith to greater than 2.2 nm.

In one embodiment, the modifier group is attached at the 5′ end or the 3′ end of the oligonucleotide of the MB. In one embodiment, the modifier group is attached within 3-7 nucleotides from the 3′ or 5′ end of the oligonucleotide of the MB in the library described herein.

In another embodiment, the modifier group is attached within 1-7 nucleotides from the 3′ or 5′ end of the oligonucleotide of the MB in the library described herein.

In one embodiment, the width of the ds nucleic acid at the point of attachment of the modifier group to the oligonucleotide of the MB in the library described herein is about 3-7 nm. In another embodiment, the width of the ds nucleic acid at the point of attachment of the modifier group to the MB oligonucleotide is about 3-5 nm.

In one embodiment, the modifier group on the oligonucleotide of the MB of the library is selected from but is not limited to the group consisting of nanoscale particles, protein molecules, organometallic particles, metallic particles and semi conductor particles. In another embodiment, the modifier group is any molecule larger than 2 nm that is not a nanoscale particle, protein molecule, organometallic particle, metallic particle or semi conductor particle.

In one embodiment, the modifier group is 3-5 nm.

In one embodiment, the modifier group on the oligonucleotide of the MB facilitates the unzipping of the ds nucleic acid when the nucleic acid is subjected to nanopore sequencing and the ds nucleic acid comprises the MBs of the library described herein.

In one embodiment, the library described herein comprises two or more species of MBs, wherein each species of MB has a distinct detectable label. In one embodiment, each species of MB complementarily hybridize to a unique nucleic acid sequence.

In one embodiment of the methods described herein, the nanopore size permits the single stranded nucleic acid to be sequenced to pass through the pore, but not the ds nucleic acid comprising the MBs of the library described herein to pass through the pore. In one embodiment of the methods described herein, the nanopore size permits the single stranded nucleic acid to translocate through the pore, but not the ds nucleic acid comprising the MBs of the library described herein.

In one embodiment of the methods described herein, the pore is larger than 2 nm. In another embodiment of the methods described herein, the pore is larger than 2.2 nm.

In one embodiment, the pore is larger than 2 nm but smaller than the width (D3) of the ds nucleic acid at the point of attachment of the modifier group to the oligonucleotide of the MB. In another embodiment, the pore is larger than 2.2 nm but smaller than the width (D3) of the ds nucleic acid at the point of attachment of the modifier group to the oligonucleotide of the MB.

In another embodiment of the methods described herein, the width (D3) of the ds nucleic acid at the point of attachment of the modifier group to the oligonucleotide of the MB is greater than 2.2 nm.

In one embodiment of the methods described herein, D1 (width of the pore) is greater than 2 nm. In another embodiment, D1 is greater than 2.2 nm.

In one embodiment of the methods described herein, D1 is 3-6 nm.

In one embodiment of the methods described herein, D3, the width of the ds nucleic acid at the point of attachment of the modifier group to the oligonucleotide of the MB, is greater than 2 nm. In another embodiment, D3 is greater than 2.2 nm.

In one embodiment of the methods described herein, D3 is about 3-7 nm.

In one embodiment of the methods described herein, the width (D3) of the ds nucleic acid at the point of attachment of the modifier group to the oligonucleotide of the MB is about 3-5 nm.

In one embodiment of the methods described herein, the width (D3) of the ds nucleic acid at the point of attachment of the modifier group to the MB oligonucleotide is greater than the width of the opening (D1) of nanopore, whereby as the ds nucleic acid attempts to pass through the nanopore opening under the influence of an electric field, the modifier group blocks the MB oligonucleotide on the ds nucleic acid from entering the opening, resulting in strand separation and the oligonucleotide of the MB is unzipped from the ds nucleic acid while the single stranded nucleic acid passes through the pore.

In one embodiment of the methods described herein, the binding affinity between the hybridized single stranded nucleic acid and MBs is less than the binding affinity of the modifier group and the oligonucleotide of the MB, whereby the bond between the single stranded nucleic acid and MBs but not the bond between the modifier group and the oligonucleotide of the MB becomes broken as the ds nucleic acid attempts to pass through the opening of the nanopore under the influence of an electric field. In one embodiment, the bond between the single stranded nucleic acid and MBs is a non-covalent hydrogen bond. In one embodiment, the bond between the modifier group and the oligonucleotide of the MB is a covalent bond. In one embodiment, the bond between the single stranded nucleic acid and MBs is a non-covalent hydrogen bond and the bond between the modifier group and the oligonucleotide of the MB is a non-covalent bond such as ionic and hydrophobic interactions. In one embodiment, the hydrogen bonds between the hybridized single stranded nucleic acid and MBs are weaker than the ionic and/or hydrophobic interactions between the modifier group and the oligonucleotide of the MB.

In one embodiment of the methods described herein, the nucleic acid to be sequenced is a DNA or an RNA.

### BRIEF DESCRIPTION OF THE DRAWINGS

FIG. 1a is a schematic illustration of the two steps in the DNA unzipping dependent sequencing methodology. First, bulk biochemical conversion of each nucleotide of the target DNA sequence to a known oligonucleotide having a known sequence, followed by hybridization with molecular beacons. Threading of the DNA/beacon complex through a nanopore allows optical detection of the target DNA sequence.

FIG. 1b is a schematic illustration of the parallel readout scheme. Each pore has a specific location in the visual field of the EM-CCD and therefore enables simultaneous readout of an array of nanopores.

FIG. 2a shows the three steps of the circular DNA conversion procedure (CDC). The 5′ template terminal nucleotide and its code are color coded “C”—purple, “A”—grey, “T”—red and “G”—blue. The colors have been changed to grey scale here.

FIG. 2b shows the analysis of the converted DNA after the CDC procedure. Left panel: a denaturing gel demonstrating successful ligation of probes to all four templates. Lanes A, T, C, and G denote respective 5-end nucleotides for the four templates, while R is the reference lane containing two ssDNA molecules, 100-nt, and 150-nt in length. Right panel: Using sequence specific fluorescent oligonucleotides, the gel shows that the first nucleotides of all four templates were successfully converted and that no by-products result from this process.

FIG. 3a shows the representative events of unzipping 1-bit and 2-bit complexes using sub 5 nm pores in an electro/optical detection of bulky group unzipping experiment. Electrical current is in black traces on the top of each panel, while the optical signal are light grey lower traces in each panel, top panel shows traces for the 1-bit samples and the lower panel shows traces for the 2-bit samples, respectively.

FIG. 3b shows histograms (n>600 for each sample) indicating that most complexes in the 1-bit sample (dark grey) produce one photon burst, while most complexes in the 2-bit sample (light grey) produce two photon bursts.

FIG. 3c shows histograms for experiments similar to those of FIG. 3b, but binned into one burst pulses, two burst pulses and 3+ burst pulses.

FIG. 4a shows the accumulated photon intensity obtained for a two-color unzipping experiments with A647 (red) and A680 (blue) fluorophores. The colors of the data have been changed to grey scale here. A single, prominent peak is observed in each channel, indicating pore location as imaged on the EM-CCD. The R values, the ratios of fluorescent intensity measured in Channel 1 vs. Channel 2, are 0.2 and 0.4 for the two fluorophores.

FIG. 4b shows the electro/optical signals for representative unzipping events with A647 (top) and A680 (bottom).

FIG. 4c shows the accumulating hundred of traces for each sample yielded R=0.20±0.06 and 0.40±0.05 for A647 and A680 respectively.

FIG. 5a shows the optical nanopore nucleobase identification using two fluorophores. Two different colors were used to enable the construction of 2-bit samples which correspond to all four DNA nucleobases. The colors of the data have been changed to grey scale here.

FIG. 5b shows the R distribution generated with >2000 events reveals two modes at 0.21±0.05 and 0.41±0.06, which correspond to the A647 and A680 fluorophores respectively, in excellent agreement with control studies.

FIG. 5c shows the representative intensity-corrected fluorescence traces of individual two-color two-bit unzipping events, with the corresponding bit called, base called and certainty score indicated above the event. The intensities in the two channels were corrected automatically by a computer code, after each bit is called using a fixed threshold R value.

FIG. 6a shows the feasibility of multi-pore detection of DNA unzipping events. The surface plots depicting accumulated optical intensity clearly indicate the locations of one (left), two (middle), and three (right) nanopores as imaged by the EM-CCD.

FIG. 6b shows four representative traces display the concurrent unzipping at two different pores. Electrical current traces (black, top trace) do not contain information on pore location, while optical traces (three lower traces) allow establishment of the location of the unzipping event.

FIG. 7 is a denaturing gel image showing the conversion of a DNA template molecule (with a C at the 5′ end). The image shows both the circularized conversion product (lane E) as well as the linearized product (lane D). Lane A is the DNA template before conversion. Included in the gel are two reference molecules, linear 150mer and circular 150 mer, lanes B and C respectively.

FIG. 8a shows the emission spectra for the two complexes containing ATTO647N dye. The top curve is the measured normalized spectrum for the molecule containing a hybridized ATTO647N beacon, while the bottom curve is the measured spectrum for the molecule containing both a hybridized ATTO647N beacon as well as a BHQ-2 quencher beacon. The inset to the figure shows schematically the complexes used.

FIG. 8b shows the emission spectra for the two complexes containing ATTO680 dye. The top curve is the measured spectrum for the molecule containing a hybridized ATTO680 beacon, while the bottem curve is the measured spectrum for the molecule containing both a hybridized ATTO680 beacon as well as a BHQ-2 quencher beacon. The inset to the figure shows schematically the complexes used.

FIG. 9 shows a schematic diagram of nanopore unzipping of a double-stranded nucleic acid with modified molecular beacons that have modifier/bulky groups linked thereon.

FIG. 10 shows the general features of one embodiment of a molecular beacon in solution and is not complementarily hybridized with a target nucleic acid. The target nucleic acid is the converted nucleic acid from the nucleic acid to be sequenced.

FIGS. 11A-11C illustrate exemplary three different conjugation schemes for linking a peptide to molecular beacons.

FIG. 11A shows a streptavidin-biotin linkage in which a molecular beacon is modified by introducing a biotin-dT to the quencher arm of the stem through a carbon-12 spacer. The biotin-modified peptides are linked to the modified molecular beacon through a streptavidin molecule, which has four biotin-binding sites.

FIG. 11B shows a thiol-maleimide linkage in which the quencher arm of the molecular beacon stem is modified by adding a thiol group which can react with a maleimide group placed to the C terminus of the peptide to form a direct, stable linkage.

FIG. 11C shows a cleavable disulfide bridge in which the peptide is modified by adding a cysteine residue at the C terminus which forms a disulfide bridge with the thiol-modified molecular beacon.

### DETAILED DESCRIPTION OF THE INVENTION

Unless otherwise explained, all technical and scientific terms used herein have the same meaning as commonly understood by one of ordinary skill in the art to which this disclosure belongs.

Unless otherwise stated, the present invention was performed using standard procedures known in the art, e.g., as described, in Current Protocols in Protein Science (CPPS) (John E. Coligan, et. al., ed., John Wiley and Sons, Inc.) which is all incorporated by reference herein in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

Embodiments of the present invention are based on an exemplary illustration that a modification to the molecular beacons (MBs) used with nanopore unzipping-dependent sequencing of nucleic acids such as DNA and RNA.

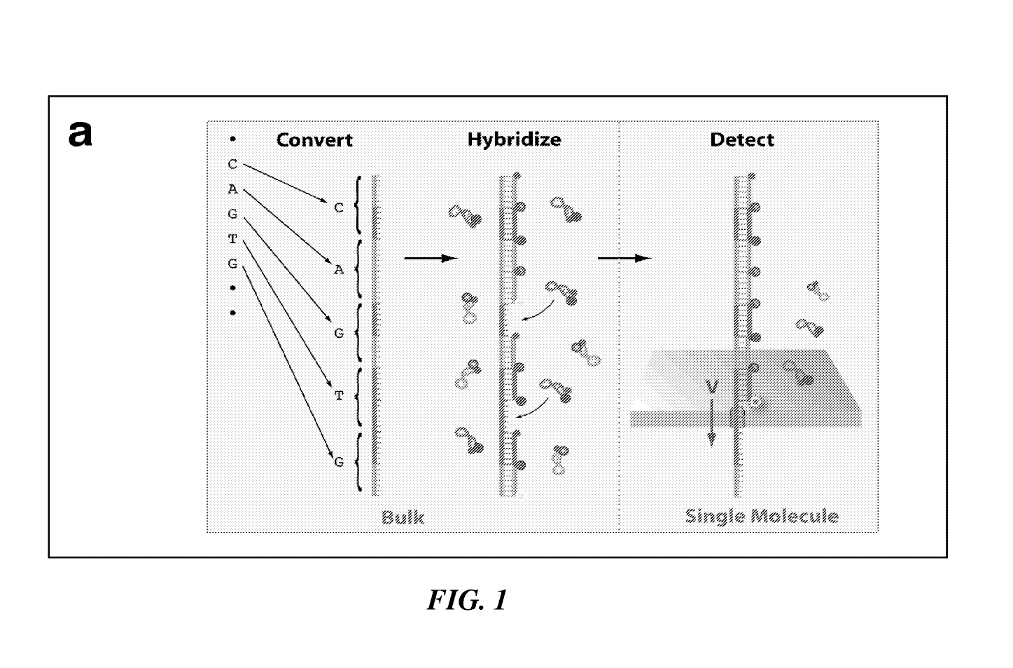

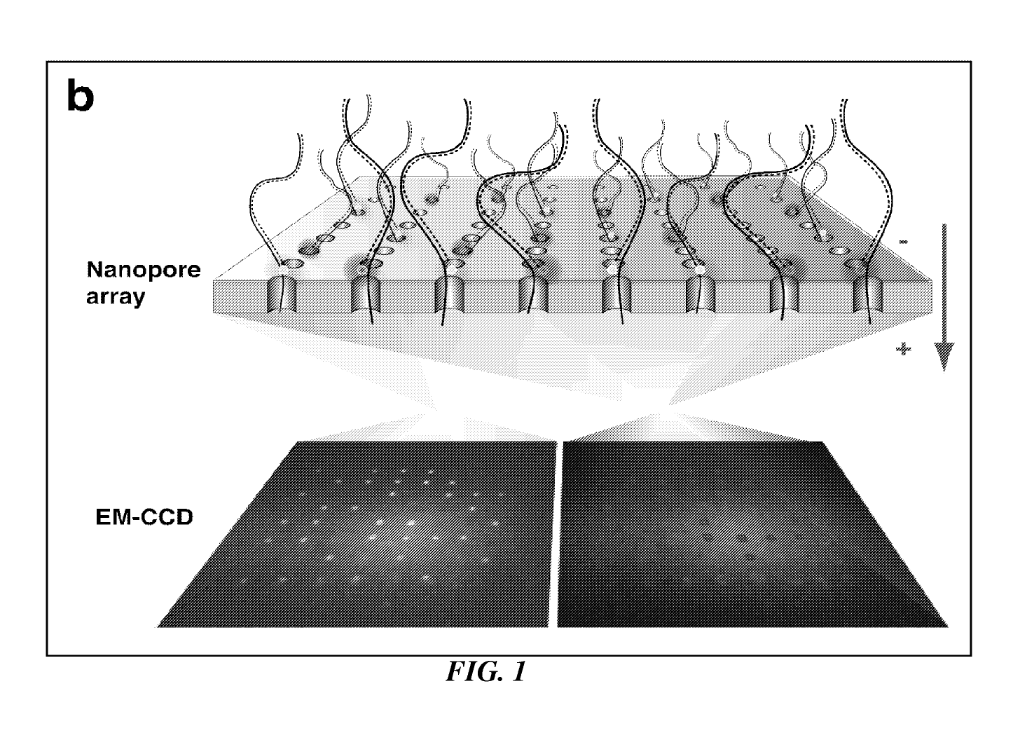

In nanopore unzipping-dependent sequencing of nucleic acids, the unzipping of a double-stranded (ds) DNA is necessary to elicit signals from the MBs comprising the dsDNA. The temporal sequence of elicited signals from the MBs corresponds to the sequence of the nucleic acid being sequenced. The size of the nanopore is used to unzip the dsDNA is limited to less than the width of a standard dsDNA that is not attached or conjugated with any extraneous molecules, the width of which is approximately 2.2 nm. Pore sizes that are about 1.5 but less than 2.2 nm can unzip a dsDNA when the dsDNA attempts to pass through the pore under the influence of an electric field, i.e. the two strands of DNA separates, and one strand passes through the pore while the other complementary strand comprising multiple non-covalently linked MBs are sequentially and temporally detected and left behind (See FIG. 1a). A pore size any larger than 2.2 nm would not facilitate the unzipping event which is necessary for eliciting signals from the MBs, wherein the elicited signals correspond to the sequence of the DNA being sequenced. A pore size any larger than 2.2 nm would simply allow the dsDNA to pass through the pore without any strand separation. In the ds DNA configuration, the hybridized MBs do not elicit any signal.

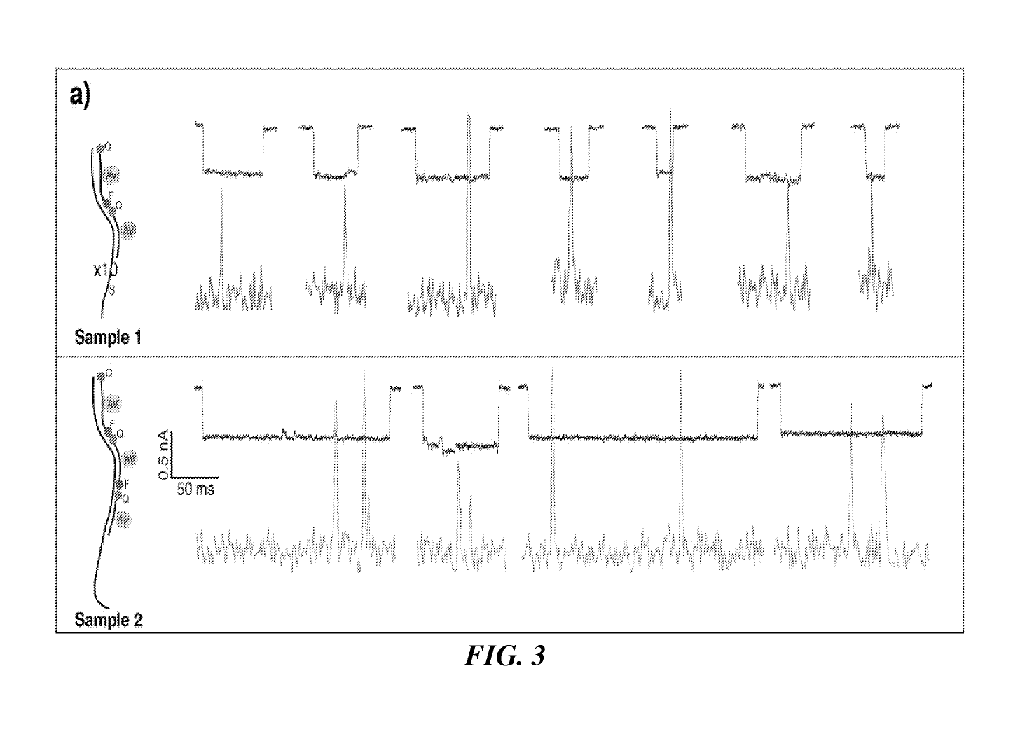

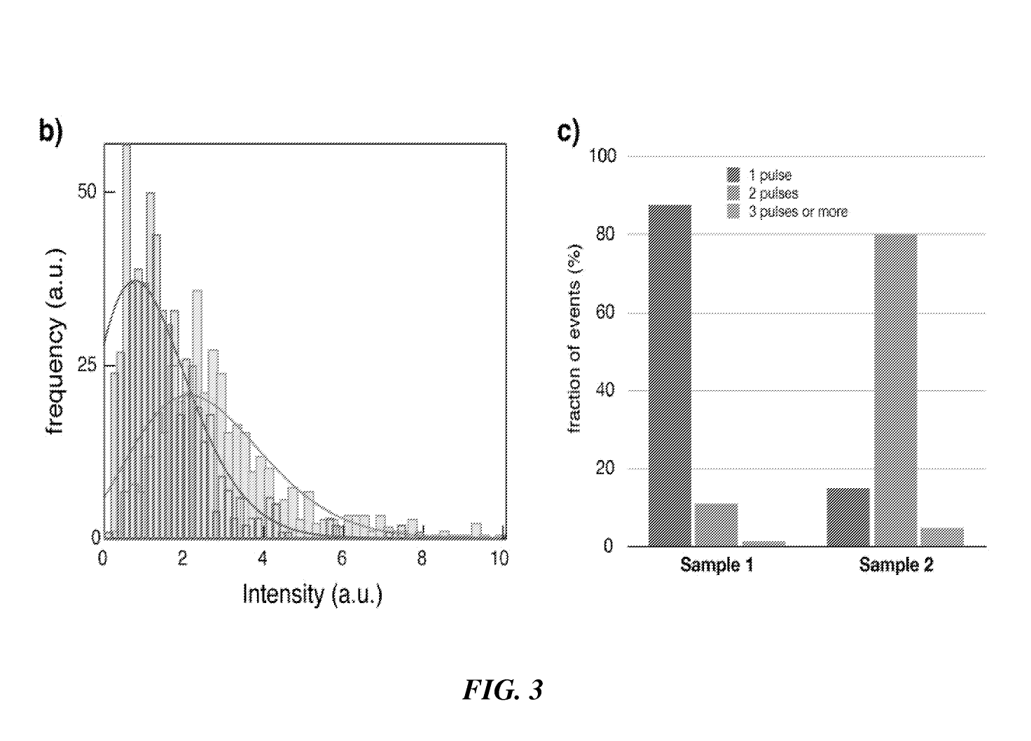

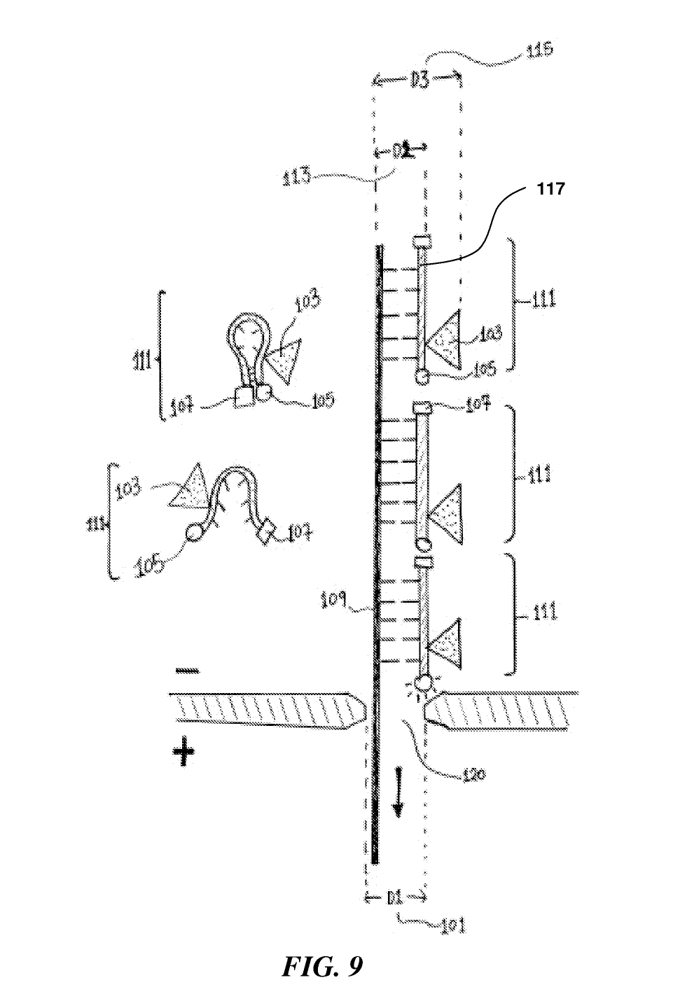

The inventors have circumvented this pore size limitation by increasing the width of the dsDNA that attempts to pass through the nanopore during sequencing, specifically by attaching a modifier group to the MBs. As schematically shown in FIG. 9, the modifier group 103 adds bulk to the MBs 111 such that the ds nucleic acid formed by a single stranded nucleic acid 109 with the modified MBs 111 have a larger width D3 115 when compared to the width D2 113 of a ds nucleic acid formed with MBs that are not modified. As a result, pore width D1 101 larger than ˜2.2 nm can be used for the unzipping event and thus sequencing, as long as the pore width D1 101 is smaller than the width of the dsDNA at the point of attachment of the bulky modifier group on the MBs, D3 115. As proof-of-concept, the inventors biotinylated a MB and attached an avidin (4.0×5.5×6.0 nm)²° to the biotinylated MB. They successfully used nanopores of 3-6 nm for unzipping the dsDNA comprising the avidin-biotinylated MBs and eliciting signals from these avidin-biotinylated MBs (FIG. 3a). Moreover, the inventors also showed that such modifications can be applied to unzipping dsDNA comprising two different species of MBs (FIG. 3a) as shown in the ‘2-bit’ experiment, where the two species of MBs are labeled with different fluorophores, e.g., one species of MB is labeled with a fluorophore that emits red fluorescence and the second species of MB is labeled with another fluorophore that emits blue fluorescence.

Since it is difficult to get consistent results when fabricating nanopores with sizes ˜2 nm or less, especially in mass production fabrication, one advantage of the disclosed modification is that larger pore sizes can be used for the nanopore based DNA sequencing that relies on the unzipping of dsDNA. This modification in turn facilitates large scale fabrication of nanopore arrays which paves the way for a straightforward method for multi-pore detection. Another advantage is that the larger pore size increase the capture rate of dsDNA by at least 10 folds and this also favors multi-pore detection in arrays¹³.

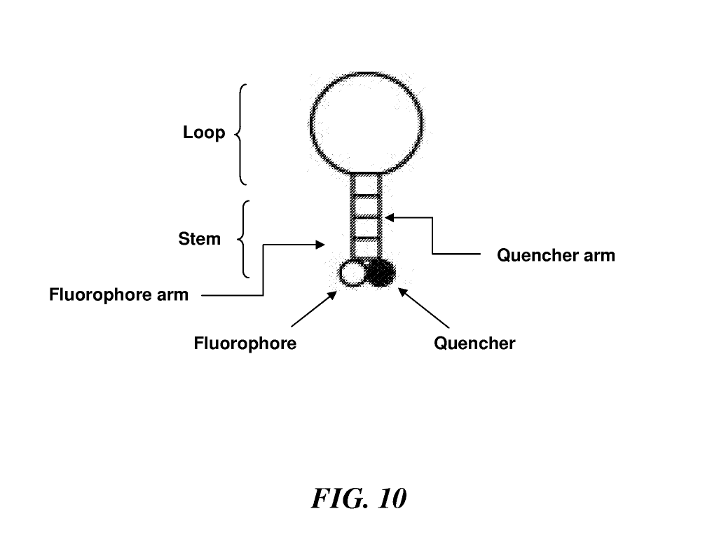

Accordingly, disclosed herein is a library of molecular beacons (MBs) for nanopore unzipping-dependent sequencing of nucleic acids, the library comprising a plurity of MBs wherein each MB comprises an oligoucleotide that comprises (1) a detectable label, (2) a detectable label blocker; and 3) a modifier group; wherein the MB is capable of sequence-specific complementary hybridization to a defined sequence that is representative of an A, U, T, C, or G nucleotide in a single-stranded nucleic acid to form a double-stranded (ds) nucleic acid. A schematic diagram of a typical MB of one eembodiment is shown is FIG. 10. In one embodiment, the oligonucleotide of the MB comprises two affinity arms. In one embodiment, the oligonucleotide of the MB comprises a 5′ affinity arm and a 3′ affinity arm. In one preferred embodiment, the oligonucleotide of the MB comprises a 5′ fluorophore arm and a 3′ quencher arm. In one embodiment, the modifier group is a quadriplex DNA. In one embodiment, the quadriplex DNA is part of and within the oligonucleotide of the MB described herein.

In one embodiment, provided herein is a method of unzipping a double-stranded (ds) oligonucleotide for nanopore unzipping-dependent sequencing of nucleic acids, the method comprising: (a) hybridizing the library of molecular beacons (MBs) described herein to a single stranded nucleic acid to be sequenced by the method, thereby forming a double stranded (ds) nucleic acid with a width of D3, which is formed by the presence of the modifier group on the MBs, wherein the single stranded nucleic acid to be sequenced is a polymer comprising defined sequences representative of A, U, T, C or G; (b) contacting the ds nucleic acid formed in step a) with an opening of a nanopore with a width of D1, wherein D3 is greater than D1; and (c) applying an electric potential across the nanopore to unzip the hybridized MBs from the single stranded nucleic acid to be sequenced.

In another embodiment, provided herein is a method for determining the nucleotide sequence of a nucleic acid comprising the steps of: (a) hybridizing the library of molecular beacons (MBs) of described herein to a single stranded nucleic acid to be sequenced, thereby forming a double stranded (ds) nucleic acid with a width of D3, which is formed by the presence of the modifier group, wherein the single stranded nucleic acid to be sequenced is a polymer comprising defined sequences representative of A, U, T, C or G; (b) contacting the ds nucleic acid formed in step a) with an opening of a nanopore with a width of D1, wherein D3 is greater than D1; and (c) applying an electric potential across the nanopore to unzip the hybridized MBs from the single stranded nucleic acid to be sequenced; and (d) detecting a signal emitted by a detectable label from each MB at the pore, as the MB separate from the ds nucleic acid as it occurs. The temporal sequence of the signal emitted corresponds to the sequence of the single stranded nucleic acid.

In one embodiment of this method of determining the nucleotide sequence of a nucleic acid, the method comprises converting a nucleic acid to be sequence to a representative single stranded nucleic acid that is hybridized by the library of MBs.

In one embodiment, the method for determining the nucleotide sequence of a nucleic acid further comprises decoding the sequence of detected signals to derive the actual nucleotide base sequence of the nucleic acid.

It is encompassed that the library and methods described herein can be used in any situations wherein the sequence of any nucleic acid or oligonucleotide is desired, e.g., detection of mutations, DNA fingerprinting, single nucleotide polymorphism, and whole genome sequencing of an organism.

A MB, as it is generally known in the art, is an oligonucleotide hybridization probe that forms a stem-and-loop structure (see FIG. 10) and is used to report the presence of specific nucleic acids in solutions. The stem-and-loop structure is also known in the art as a hairpin or hairpin loop. MBs are also referred to as molecular beacon probes. As exemplary and should not be construed as limiting, the general design and features of a typical MB oligonucleotide probe are as follows (see: FIG. 10): The MB can be of various length, e.g., about 15-35 nucleotides long. In embodiments where there is a quadriplex portion of DNA within the MB, the length of the MB can be longer, e.g., up to 60 nucleotides long. In one embodiment, the middle portion forms the “loop”, comprising 5-25 nucleotides that are complementary to a specific target DNA or RNA or oligonucleotide. As used in the context of a MB, the “target nucleic acid’, “target DNA”, “target sequence”, “target RNA” or “target oligonucleotide” is a nucleic acid that the MB can complemenarily hybridize with, i.e., “base-pair” with, base on the Watson-Crick type hybridization. In one embodiment, there are at least two nucleotides at each end of the MB that are complementary to each other, i.e., can “base-pair” with each other. These two nucleotides at each end or “affinity arm” of the MB anneal together and forms the ‘stem” of MB, producing the stem-and-loop structure when the MB is not hybridized with its target nucleic acid. The stem-and-loop structure is typically 2-7 nucleotides long at the sequences at both the ends are complementary to each other.

In one embodiment, a dye or a detectable label is attached towards the 5′ end/arm of the MB, commonly termed the 5′ fluorophore that fluoresces in presence of a complementary target. In one embodiment, a quencher dye or a detectable label blocker is covalently attached to the 3′ end/arm of the MB, commonly termed the 3′ quencher. When the beacon is in the closed loop shape, the quencher prevents the fluorophore from emitting light. Generally, MBs form stem-and-loop shaped molecules with an internally quenched fluorophore whose fluorescence is restored when they bind to a target nucleic acid sequence. Below is an example of a MB:

Fluorophore at 5′ end; 5′-GCGAGCTAGGAAACACCAAAGATGATATTTGCTCGC-3′-DABCYL (SEQ. ID. NO:2). DABCYL a non-fluorescent chromophore, can serves as a universal quencher for any fluorophore in MBs.

In another embodiment, the MBs have no stem-loop structure. There are no nucleotides at each end of the MB that are complementary to each other, hence no stem-loop structure are formed. In one embodiment, the MBs of the library do not form a stem-loop structure.

In one embodiment, the MB is an oligonucleotide with a detectable label. In a further embodiment, the MB is an oligonucleotide with a detectable label and a detectable label blocker.

In one embodiment, the MBs do not fluoresce when they are free in solution under suitable conditions of temperature and ionic strength (e.g., below the Tₘ of the stem-loop structure). When MBs hybridize to a nucleic acid that is complementary to the MB probe or loop region, the MB undergo a conformational change that enables them to fluoresce brightly. In the absence of a complementary nucleic acid, the probe is dark, because the stem places the fluorophore so close to the fluorescence quencher that the fluorophore and quencher transiently share electrons, eliminating the ability of the fluorophore to emit fluoresce. When the probe encounters a suitable complementary nucleic acid molecule, it forms a probe-target hybrid that is longer and more stable than the stem hybrid. The rigidity and length of the probe-target hybrid precludes the simultaneous existence of the stem hybrid. Consequently, the MB undergoes a spontaneous conformational reorganization that forces the stem hybrid to dissociate and the fluorophore and the quencher to move away from each other, thereby allowing the fluorophore to emit fluorescence upon excitation with a suitable light source,

In one embodiment, the entire oligonucleotide of a MB is complementary to a target nucleic acid. For the unzipping DNA nanopore method, the target nucleic acid would be the specific nucleic acid sequence or a polymer that is representative of A, U, T, C or G.

In one embodiment, the 3′ and 5′ affinity arms of the oligonucleotide of the MB are complementary to each other in the absence of a target nucleic acid. In the presence of a target nucleic acid, the 3′ and 5′ affinity arms of the oligonucleotide of the MB are complementary to the target nucleic acid. The target nucleic acid for the MBs of the library described herein is a nucleic acid sequence or a polymer that is representative of A, U, T, C or G. In the absence of the target nucleic acid sequence, the 3′ and 5′ affinity arms of the MB anneal and form the stem of the MB stem-and-loop structure.

In some embodiments, the entire oligonucleotide of a MB is a sequence having 4 to 60 nucleotides. In other embodiments, the entire oligonucleotide of a MB is a sequence having 8 to 32 nucleotides. For instance, a library of MBs can be such that all the MBs are 8 nucleotides long. In other instances, the library of MBs can be such that all the MBs are 16 nucleotides long, 32 nucleotides long, 45 or 60 nucleotides long. In one embodiment, a library of MBs comprises at least two species of MBs, wherein the two species have different oligonucleotide length of the MBs. For example, one species can be 8 nucleotides long and the other species can be 16 nucleotides long for a library with only two species.

In certain embodiments, the “loop” region complementarily hybridizes to the target nucleic acid, e.g., a nucleic acid sequence or a polymer that is representative of A, U, T, C or G. In certain embodiments, the “loop” region complementarily hybridizes with a sequence having 4 to 32 nucleotides on the target nucleic acid.

In certain embodiments, the affinity arm of the stem of the MB also complementarily hybridizes with a target sequence having 4 to 25 nucleotides.

In one embodiment, the oligonucleotide of a MB comprises a quadruplex portion. G-quadruplexes are higher-order DNA and RNA structures formed from G-rich sequences that are built around tetrads of hydrogen-bonded guanine bases. Such quadruplex sequences are well known in the art, e.g., as described by Burge, S. et al., Nucleic Acids Research, 2006, 34:5402-5415; Borman, S., Chemical and Engineering News, 2007, 85:12-17; Hammond-Kosack and K. Docherty, FEB s Letters, 1992, 301:79-82; and Chen C Y et al., Sex Transm. Infect., 2008, 84:273-6. These references are incorporated herein by reference in their entirety. Therefore, one skilled in the art can design and incorporate a quadruplex into the MBs of a library. In one embodiment, the quadruplex portion does not complementary hybridize with a target nucleic acid sequence or a polymer representative of A, U, T, C or G. In one embodiment, the quadruplex portion serves as the bulky modifier group. In one embodiment, the quadruplex portion of the MB is found at the 3′ or 5′ ends of the oligonucleotide of the MB. In one embodiment, the quadruplex portion of the MB is located at 2-7 nucleotides from the 3′ or 5′ ends of the oligonucleotide of the MB. In another embodiment, the quadruplex portion of the MB is located at 1-7 nucleotides from the 3′ or 5′ ends of the oligonucleotide of the MB.

In reference to an oligonucleotide being capable of sequence-specific complementary hybridization or complementary to a sequence means the oligonucleotide forms the canonical Watson and Crick nucleotide base pairing by hydrogen bonds with the sequence, wherein adenine (A) forms a base pair with thymine (T), as does guanine (G) with cytosine (C) in DNA. In RNA, thymine is replaced by uracil (U).

In certain embodiments for the purposes of nanopore unzipping-dependent sequencing, the nucleic acid that is to be sequenced is first converted to a representative sequence. The representative sequence functions to magnify each single base in the nucleic acid to be sequence into a larger sequence. The larger representative sequence is made up of blocks of sequence, also termed as codes or block sequence, which are defined, unique and fixed for each base A, T C, G, and U. For example, an “A” in a nucleic acid to be sequence is represented by an expanded 10-mer block sequence of ATTTATTAGG (SEQ. ID. NO. 3), an “T” is represented by an expanded 10-mer block sequence of CGGGCGGCAA (SEQ. ID. NO. 4), an “C” is represented by an expanded 10-mer block sequence of CCTTTCCTTA (SEQ. ID. NO. 5), and an “G” is represented by an expanded 10-mer block sequence of AGCGCCGAAC (SEQ. ID. NO. 6). As a result, a nucleic acid having a “TGGCA” sequence will be converted to a representative sequence CGGGCGGCAA-AGCGCCGAAC-AGCGCCGAAC-CCTTTCCTTA-ATTTATTAGG (SEQ. ID. NO. 7) which comprises five 10-mer block sequences. Since the bases A, T, C, G are represented by four unique 10-mer block sequences in this example, this is a uni- or single code system of sequence conversion. When a base is represented by a pair of block sequences, it is a binary coded system of sequence conversion. For example, the binary code is two unique 10-mer block sequences: ATTTATTAGG (SEQ. ID. NO. 3) and CGGGCGGCAA (SEQ. ID. NO. 4), and they can be referred to as code “0” and “1” respectively. Each base is represented by a pair of block sequence, e.g., “A” is represented by “0,1” or ATTTATTAGG-CGGGCGGCAA (SEQ. ID. NO. 8), “T” is represented by “0,0” or ATTTATTAGG-ATTTATTAGG (SEQ. ID. NO. 9), “C” is represented by “1,0” or CGGGCGGCAA-ATTTATTAGG (SEQ. ID. NO. 10), and “G” is represented by “1,1” or CGGGCGGCAA-CGGGCGGCAA (SEQ. ID. NO.11). The sequential arrangement of the pair of block sequences or codes is important, meaning that “0,1” is not the same an “1,0” because “0,1” codes for an A while “1,0” codes for a “C” in the above example. Therefore, when using a binary code system described herein, a nucleic acid having a “GATGGCA” sequence will be converted to a binary code of (11)-(01)-(00)-(11)-(11)-(10)-(01) or a representative sequence (CGGGCGGCAA-CGGGCGGCAA)-(ATTTATTAGG-CGGGCGGCAA)-(ATTTATTAGG-ATTTATTAGG)-(CGGGCGGCAA-CGGGCGGCAA)-(CGGGCGGCAA-CGGGCGGCAA)-(CGGGCGGCAA-ATTTATTAGG)-(ATTTATTAGG-CGGGCGGCAA) (SEQ. ID. NO. 12). Detail descriptions of the conversion of a nucleic acid to be sequence and the coded system for conversion can be found in Soni and Meller (2007)²⁹, Meller et al., 2009 (U.S. Patent Application publication 2009/0029477), and Meller and Weng (PCT Application No. PCT US 2009/034296). These references are incorporated herein by reference in their entirety.

In one embodiment, the define sequence that is representative of an A, U, T, C, or G nucleotide in a single-stranded nucleic acid comprises block sequences, wherein the block sequences are representative of an A, U, T, C, or G nucleotide in a single-stranded nucleic acid.

In one embodiment, the oligonucleotide of the MB is complementary to the block sequences of the define sequence that is representative of an A, U, T, C, or G nucleotide in a single-stranded nucleic acid.

In one embodiment, the library comprises several species of MBs, wherein there is at least one species of MB for each block sequence that is representative of an A, U, T, C, or G nucleotide in a single-stranded nucleic acid. Each species has a distinct detectable label that is different from that of the other species in the library. For example, if there are four species of MBs in the library, then there are four distinct detectable labels, e.g., red, green, blue and yellow for fluorophore as detectable labels. Each species also has a distinct oligonucleotide sequence that is different from that of the other species of MBs in the library. For example, if there are four species of MBs in the library, then there are four distinct oligonucleotide sequences, e.g., ATTTATTAGG (SEQ. ID. NO. 3), CGGGCGGCAA (SEQ. ID. NO. 4), CCTTTCCTTA (SEQ. ID. NO. 5), and AGCGCCGAAC (SEQ. ID. NO. 6) in the MBs of the library.

In the embodiment where a uni- or single code system of sequence conversion is utilized, the library comprises at least four species of MBs. In one embodiment, the library comprises at least two species of MBs and up to four species of MBs, wherein each species has a different fluorophore and a distinct sequence. In one embodiment, the library comprises at least two species of MBs and up to six species of MBs, wherein each species has a different fluorophore and a distinct sequence. In one embodiment, the library comprises up to eight species of MBs wherein each species has a different fluorophore and a distinct sequence. In one embodiment, the library comprises four species of MBs, e.g., four different types of MBs with each type having a different fluorophore and a distinct sequence.

In the embodiment where a binary code system of sequence conversion is utilized, the library comprises at least two species of MBs, e.g., two different types of MBs with one type having a fluorophore and unique sequence for code “0” and the other type of MB having a different fluorophore and unique sequence for code “1”. In one embodiment, the library comprises two species of MBs. Each species of MBs has it own unique oligonucleotide sequence that can complementary hybridize with its specific block sequence.

In one embodiment, each species of MB has a distinct detectable label. In one embodiment, each species of MB has the same detectable label blocker. In another embodiment, each species of MB has the same modifier group.

In one embodiment, the library described herein comprises at least two distinct detectable labels on the MBs therein, wherein only one detectable label is on each MB. In one embodiment, the library described herein comprises two distinct detectable labels on the MBs therein, wherein only one detectable label is on each MB. In one embodiment, the library described herein comprises four distinct detectable labels on the MBs therein, wherein only one detectable label is on each MB. For example in the binary code system described herein, a library will have two species of MBs, one first species of MBs has sequences that can complement the “0” code which has the sequence of ATTTATTAGG (SEQ. ID. NO. 3) and a second species of MBs of the library has sequences that can complement the “1” code which has the sequence of CGGGCGGCAA (SEQ. ID. NO. 4). In one embodiment, there are two or more species of MBs, wherein each species of MB has a distinct detectable label. For example, a library comprises two species of MBs, one first species of MBs have ATTO647N fluorophore as a detectable group and the second species of MBs of the library has ATTO488 fluorophore as a detectable group (see Example section). Both ATTO647N-MBs and ATTO488-MBs have the same detectable label blocker, a quencher BHQ-2. In addition, both ATTO647N-MBs and ATTO488-MBs have the same modifier group, avidin-biotin.

In nanopore unzipping-dependent sequencing, a plurality of MBs is bound in a tandem arrangement on to a sequence forming a ds polymer. For example using the binary coded system described herein, a sequence having the binary code of (11)-(01)-(00)-(11)-(11)-(10)-(01) or a representative sequence (CGGGCGGCAA-CGGGCGGCAA)-(ATTTATTAGG-CGGGCGGCAA)-(ATTTATTAGG-ATTTATTAGG)-(CGGGCGGCAA-CGGGCGGCAA)-(CGGGCGGCAA-CGGGCGGCAA)-(CGGGCGGCAA-ATTTATTAGG)-(ATTTATTAGG-CGGGCGGCAA) (SEQ ID NO: 12) will have 14 MBs complementarily hybridized in a tandem arrangement with the sequence to form a ds polymer. The tandem arrangement of the MBs is such that the 3′ quencher of a preceding MB quenches by the fluorescence of the subsequent MB's 5′ fluorophore (see FIG. 1). Detailed disclosure of the nanopore unzipping-dependent sequencing using MBs are described in Soni and Meller (2007)²⁹ and in U.S. Patent Application Publication No. 2009/0029477, all of which are incorporated herein by reference in their entirety.

In one embodiment, the MB is an oligonucleotide such as a DNA and an RNA. In one embodiment, the oligonucleotide is a single stranded oligonucleotide. In another embodiment, the MB is an oligonucleotide such as glycol nucleic acid (GNA), locked nucleic acid (LNA), peptide nucleic acid (PNA), threose nucleic acid (TNA), and Morpholino. In one embodiment, the oligonucleotide of the MB comprises a nucleic acid selected from but is not limited to a group consisting of deoxyribonucleic acid (DNA), ribonucleic acid (RNA), glycol nucleic acid (GNA), peptide nucleic acid (PNA), locked nucleic acid (LNA), threose nucleic acid (TNA) and phosphorodiamidate morpholino oligo (PMO/Morpholino). In another embodiment, the MB is a chimeric oligonucleotide; e.g., comprises a mixture or combination of DNA, RNA, GNA, PNA, LNA, TNA and Morpholino. Examples include but are not limited to DNA/RNA chimeric MBs, DNA/LNA chimeric MBs, and RNA/PNA chimeric MBs.

In one embodiment, the oligonucleotide of the MB comprises 4-60 nucleotides. In other embodiments, the oligonucleotide of the MB comprises 7-32 nucleotides, 4-25 nucleotides, 4-16 nucleotides, 4-32 nucleotides, 7-16 nucleotides or 7-25 nucleotides. In one embodiment, the oligonucleotide comprises 8-16 nucleotides. In some embodiments, the oligonucleotide comprises 7, 8, 16 or 32 nucleotides. In one embodiment, all the species of MBs in the library have oligonucleotides of the same number of nucleotides. In another embodiment, the species of MBs in the library have oligonucleotides having a number of nucleotides. In one embodiment, the nucleotide is selected from a group consisting of deoxyribonucleic acid (DNA), ribonucleic acid (RNA), glycol nucleic acid (GNA), peptide nucleic acid (PNA), locked nucleic acid (LNA), threose nucleic acid (TNA) and phosphorodiamidate morpholino oligo (PMO/Morpholino). The oligonucleotides generally are at least about 6 to about 25 nucleotides, often at least about 10 to about 20 nucleotides, and frequently at least about 11 to about 16 nucleotides in length. The 16-mer and 32-mer oligonucleotide MBs described herein are exemplary and should not in any way be limiting. In some embodiments, the oligonucleotide of the MB is a polymer of nucleotide, nucleobases or monomers.

GNA is a polymer similar to DNA or RNA but differing in the composition of its “backbone”. GNA is not known to occur naturally. While DNA and RNA have a deoxyribose and ribose sugar backbone, the GNA's backbone is composed of repeating glycerol units linked by phosphodiester bonds. The glycerol molecule has just three carbon atoms and is capable of Watson-Crick base pairing. The Watson-Crick base pairing is much more stable in GNA than its natural counterparts DNA and RNA as it requires a high temperature to melt a duplex of GNA. Examples of GNAs are the 2,3-dihydroxypropylnucleoside analogues that were first prepared by Ueda et al. (1971) Journal of Heterocyclic Chemistry 8(5), 827-9. Other GNAs polymer and their preparation and properties are disclosed in Seita et al. (1972) Die Makromolekulare Chemie, 154:255-261; Cook et al. (1995) PCT Int. Appl., WO 9518820, 126 pp.; U.S. Pat. No. 5,886,177; Acevedo and Andrews (1996) Tetrahedron Letters 37(23):3931-3934 and Zhang et al., (2005), J. Am. Chem. Soc. 127 (12): 4174-5. These references are all incorporated herein by reference in their entirety.

TNA is a polymer similar to DNA or RNA but differing in the composition of its “backbone”. TNA is not known to occur naturally. Unlike DNA and RNA which have a deoxyribose and ribose sugar backbone, respectively, TNA's backbone is composed of repeating threose units linked by phosphodiester bonds. The threose molecule is easier to assemble than ribose. TNA can specifically base pair with RNA and DNA. J Am Chem. Soc. 2005, 127:2802-3. An example of a TNA is (3′-2′)-alpha-1-threose nucleic acid. Other TNAs are described by Orgel, Leslie, 2000, Science 290 (5495): 1306-1307; Watt, Gregory, 2005, Nature Chemical Biology; and Schoning, K. et al., 2000, Science 290: 1347. These references are all incorporated herein by reference in their entirety.

PNA is an artificially synthesized polymer similar to DNA or RNA invented by Peter E. Nielsen and collegues in 1991 (Science, 254:1497). PNA's backbone is composed of repeating N-(2-aminoethyl)-glycine units linked by peptide bonds. The various purine and pyrimidine bases are linked to the backbone by methylene carbonyl bonds. PNAs are depicted like peptides, with the N-terminus at the first (left) position and the C-terminus at the right. Therefore, PNA is a DNA mimic with a pseudopeptide backbone. PNA is an extremely good structural mimic of DNA (or RNA). Since the backbone of PNA contains no charged phosphate groups, the binding between PNA/DNA strands is stronger than between DNA/DNA strands due to the lack of electrostatic repulsion. PNA oligomers are able to form very stable duplex structures with Watson-Crick complementary DNA, RNA (or PNA) oligomers, and they can also bind to targets in duplex DNA by helix invasion. (See Egholm, M., et al., (1993) Nature, 365, 566-568; Wittung, P., et al., (1994) Nature, 368, 561-563). These references are all incorporated herein by reference in their entirety.

LNA is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form of DNA or RNA. LNA nucleotides can be mixed with DNA or RNA bases in the oligonucleotide whenever desired. The locked ribose conformation enhances base stacking and backbone pre-organization. This significantly increases the thermal stability (melting temperature) of oligonucleotides (Kaur, H, et al., (2006), Biochemistry 45 (23): 7347-55). LNA nucleotides have been used to increases the sensitivity and specificity of expression in DNA microarrays, FISH probes, real-time PCR probes and other molecular biology techniques based on oligonucleotides. The synthesis of LNAs and their hybridization properties are described by Alexei A., et al., (1998), Tetrahedron 54 (14): 3607-30; You Y., et al., (2006), Nucleic Acids Res. 34 (8): e60. These references are all incorporated herein by reference in their entirety.

Morpholinos are synthetic molecules that can hybridize to complementary sequences by standard nucleic acid base-pairing. Morpholinos have nucleotide bases bound to morpholine rings instead of deoxyribose rings and linked through phosphorodiamidate groups instead of phosphates. Replacement of anionic phosphates with the uncharged phosphorodiamidate groups eliminates ionization in the usual physiological pH range, so Morpholinos are generally uncharged molecules. The entire backbone of a Morpholino is made from these modified subunits. Morpholinos are most commonly used as single-stranded oligonucleotides, though heteroduplexes of a Morpholino strand and a complementary DNA strand may be used in combination with cationic cytosolic delivery reagents.

Morpholinos are also in development as pharmaceutical therapeutics targeted against pathogenic organisms such as bacteriaor viruses and for amelioration of genetic diseases. For example, in an antisense technology, in suppression of gene expression (Moulton, Jon (2007). “Using Morpholinos to Control Gene Expression (Unit 4.30)” in Beaucage, Serge. Current Protocols in Nucleic Acid Chemistry. New Jersey: John Wiley & Sons, Inc. This reference is incorporated herein by reference in their entirety. Because of their completely unnatural backbones, Morpholinos are not recognized by cellular proteins. Nucleases do not degrade Morpholinos, nor are they degraded in serum or in cells. Morpholinos do not activate toll-like receptors and so they do not activate innate immune responses such as interferon induction or the NF-κB mediated inflammation response. Morpholinos are not known to modify methylation of DNA.

In one embodiment, the MBs of the library described herein are not attached to a solid phase carrier, such as a glass slide or a microbead. In one embodiment, the MBs of the library described herein are free in solution. In another embodiment, the MBs of the library described herein, when free in solution, assumes a “loop-stem” configuration enabling the detectable label group blocker to block the detectable group from emitting a signal in the absence of a target nucleic acid to anneal to the MB. In another embodiment, the MBs of the library described herein, when free in solution, assumes a configuration that enables the detectable label group blocker to block the detectable group from emitting a signal in the absence of a target nucleic acid to anneal to the MB. In yet another embodiment, the MBs of the library described herein, when free in solution, do not assume a “loop-stem” configuration. In one embodiment, MBs do not fluoresce when they are free in solution under suitable conditions of temperature and ionic strength (e.g., below the Tₘ of the stem-loop structure).

In one embodiment, the detectable label is located on one end of the oligonucleotide of the MB and is located on the same end for all oligonucleotide of the MBs in the library, wherein the detectable label emits a signal that can be detected and/or measured when the detectable label is not inhibited by a blocker. In one embodiment, the detectable label is located at the 5′ end of the oligonucleotide of the MB. In one embodiment, the detectable label is located at the 5′ end of all oligonucleotide of the MBs in the library. In another embodiment, the detectable label is located at the 3′ end of the oligonucleotide of the MB. In one embodiment, the detectable label is located at the 3′ end of all oligonucleotide of the MBs in the library. In one embodiment, the detectable label is covalently linked to the end of one arm of the oligonucleotide of the MB, preferably the 5′ arm of the oligonucleotide. In one embodiment, the detectable label is covalently linked to the 5′ arm of the oligonucleotide. In one embodiment, the detectable label is covalently linked to the 3′ arm of the oligonucleotide of the MB.

In one embodiment, the detectable label, detectable label blocker and the modifier group on the oligonucleotide of the MB do not interfere with sequence-specific complementary hybridization of the MB with the define sequence that is representative of an A, U, T, C, or G nucleotide in a single-stranded nucleic acid.

In one embodiment, the detectable group's signal is detected optically. As used herein, “detected optically” with regards to the detectable group signal refers to the measurement of light energy which is the signal emitted by the detectable group. In one embodiment, the light energy emitted has a wavelength range of 380-760 nm. In another embodiment, the light energy emitted has a wavelength range of 700 nm-1400 nm. In another embodiment, the detectable group's signal is not detected optically.

In one embodiment, the detectable group is a fluorophore and the signal is fluorescence. MBs can be made in many different colors utilizing a broad range of fluorophores (Tyagi S, et al., Nature Biotechnology 1998; 16: 49-53). Examples of fluorophores for use with MB include but are not limited to Alexa Fluor® 350; Marina Blue®; Atto 390; Alexa Fluor® 405; Pacific Blue®; Atto 425; Alexa Fluor® 430; Atto 465; DY-485XL; DY-475XL; FAM™ 494; Alexa Fluor® 488; DY-495-05; Atto 495; Oregon Green® 488; DY-480XL 500; Atto 488; Alexa Fluor® 500; Rhodamin Green®; DY-505-05; DY-500XL; DY-510XL; Oregon Green® 514; Atto 520; Alexa Fluor® 514; JOE 520; TET™ 521; CAL Fluor® Gold 540; DY-521XL; Rhodamin 6G®; Yakima Yellow® 526; Atto 532; Alexa Fluor®532; HEX 535; VIC 538; CAL Fluor Orange 560; DY-530; TAMRA™; Quasar 570; Cy3™ 550; NED™; DY-550; Atto 550; Alexa Fluor® 555; DY-555; Alexa Fluor® 546; BMN™-3; DY-547; PET®; Rhodamin Red®; Atto 565; CAL Fluor RED 590; ROX; Alexa Fluor® 568; Texas Red®; CAL Fluor Red 610; LC Red® 610; Alexa Fluor® 594; Atto 590; Atto 594; DY-600XL; DY-610; Alexa Fluor® 610; CAL Fluor Red 635; Atto 620; DY-615; LC Red 640; Atto 633; Alexa Fluor® 633; DY-630; DY-633; DY-631; LIZ 638; Atto 647N; BMN™-5; Quasar 670; DY-635; Cy5™.; Alexa Fluor®647; CEQ8000 D4; LC Red 670; DY-647 652; DY-651; Atto 655; Alexa Fluor® 660; DY-675; DY-676; Cy5.5™675; Alexa Fluor® 680; LC Red 705; BMN™-6; CEQ8000 D3; IRDye® 700Dx 689; DY-680; DY-681; DY-700; Alexa Fluor® 700; DY-701; DY-730; DY-731; DY-732; DY-750; Alexa Fluor® 750; CEQ8000 D2; DY-751; DY-780; DY-776; IRDye® 800CW; DY-782; and DY-781; Oyster® 556; Oyster® 645; IRDye® 700, IRDye® 800; WellRED D4; WellRED D3; WellRED D2 Dye; Rhodamine Green™; Rhodamine Red™; fluorescein; MAX 550 531 560 JOE NHS Ester (like Vic); TYE™563; TEX 615; TYE™ 665; TYE 705; ODIPY 493/503™; BODIPY 558/568™; BODIPY 564/570™; BODIPY 576/589™; BODIPY 581/591™; BODIPY TR-X™; BODIPY-530/550™; carboxy-X-Rhodamine™; carboxynaphthofluorescein; carboxyrhodamine 6G™; Cascade Blue™; 7-Methoxycoumarin; 6-JOE; 7-Aminocoumarin-X; and 2′,4′,5′,7′-Tetrabromosulfonefluorescein cyanine dye; thiazole orange; digoxigenin; fluorescein (FAM); rhodamine x (ROX); tetrachloro-6-carboxyfluorescein (TET); tetramethylrhodamine (TAMRA); Alexa Fluor; BODIPY®; OREGON GREEN®; CASCADE BLUE®; Marina Blue®; PACIFIC BLUE™; RHODAMINE GREEN™; RHODAMINE REM and TEXAS RED® are commercially available fluorophores from Molecular Probes, Inc.

In one embodiment, the detectable label blocker is a quencher of the fluorophore. Examples of a quencher of fluorophores for use with MB include but are not limited to 3′ IOWA BLACK™ FQ, 3′ BLACK HOLE QUENCHER®-1, and 3′ Dabcyl; BHQ-1®; BHQ-2®; BBQ-650; DDQ-1; Iowa Black RQ™; Iowa Black FQ™; QSY-21®; QSY-35®; QSY-7®; QSY-9®; QXL™ 490; QXL™ 570; QXL™ 610; QXL™ 670; QXL™ 680; DNP; and EDANS.

Many combinations of quencher-fluorophore exist, each producing a unique color or fluorescence emission profile (see e.g., the World Wide Web site of molecularbeacons.org and references cited therein). The skilled artisan will recognize that individual fluorophores and quenchers are each optimally active at a particular wavelength or range of wavelengths. Therefore, a skilled artisan would know to choose fluorphore and quencher pairs such that the fluorophore's optimal excitation and emission spectra are matched to the quencher's effective range. Examples of quencher-fluorophore pairs comtemplated are: 6-FAM, HEX, or TET with 3′-Dabcyl; 5′-Coumarin or Eosin with 3′-Dabcyl; 5′-Texas Red or Tetramethylrhodamine with 3′-BLACK HOLE QUENCHER®; and EDANS and 3′-DABCYL.

In one embodiment, both the detectable label blocker and the detectable label are located at the same end of the oligonucleotide of the MBs, i.e., both on the 3′ end or both on the 5′ end of the oligonucleotide of the MBs. In one embodiment, the detectable label blocker is not located immediately next to the detectable label on the oligonucleotide of the MB. In one embodiment, the detectable label blocker and the detectable label is separated by at least 3 nucleotides or monomers on the oligonucleotide of the MB, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, or at least 25 nucleotides or monomers on the oligonucleotide of the MB.

In one embodiment, the detectable label blocker is located at one end of the oligonucleotide of the MB while the detectable label is located at the other end of oligonucleotide of the MBs. In one embodiment, the detectable label blocker is covalently linked to one arm of the oligonucleotide of the MB, preferably the 3′ arm of the oligonucleotide of the MB. In one embodiment, the detectable label blocker is covalently linked to the 3′ arm of the oligonucleotide of the MB. In another embodiment, the detectable label blocker is covalently linked to the 5′ arm of the oligonucleotide of the MB.

In one embodiment, the detectable label blocker is located at the end opposite that of the detectable label on the oligonucleotide of the MB. For example, if the detectable label blocker is located at the 5′ end of the oligonucleotide of the MB, then the detectable label is located at the 3′ end of the oligonucleotide of the same MB. In one embodiment, the detectable label blocker is covalently linked to the end of one arm of the oligonucleotide of the MB and a detectable label is covalently linked to the end of the other arm of the same oligonucleotide. In one embodiment, the detectable label blocker is covalently linked to the 3′ arm of the oligonucleotide of the MB and the detectable label is covalently linked to the 5′ arm of the same oligonucleotide. In one embodiment, the detectable label blocker is covalently linked to the 5′ arm of the oligonucleotide of the MB and the detectable label is covalently linked to the 3′ arm of the same oligonucleotide. In one embodiment, a fluorophore is covalently linked to the end of one arm of the oligonucleotide of the MB and a fluorescence quencher is covalently linked to the end of the other arm of the same oligonucleotide. In one preferred embodiment, a fluorescence quencher is covalently linked to the 3′ arm of the oligonucleotide of the MB and a fluorophore is covalently linked to the 5′ arm of the same oligonucleotide. In another preferred embodiment, the 3′ arm of the oligonucleotide of the MB refers to the 3′ end of the oligonucleotide of the MB and the 5′ arm of the oligonucleotide of the MB refers to the 5′ end of the oligonucleotide of the MB.

In certain embodiments, the detectable labels, the detectable label blocker and modifier groups are conjugated to the oligonucleotide of the MB by covalent linkage. In one embodiment, covalent linkage comprises spacers, preferably linear alkyl spacers. By “conjugated” is meant the covalent linkage of at least two molecules. The nature of the spacer is not critical. For example, fluorescence quencher such as EDANS and DABCYL can be linked via six-carbon-long alkyl spacers well known and commonly used in the art. The alkyl spacers give the detectable labels and the detectable label blocker enough flexibility to interact with each other for efficient fluorescence resonance energy transfer, and consequently, efficient quenching. The chemical constituents of suitable spacers will be appreciated by persons skilled in the art. The length of a carbon-chain spacer can vary considerably, e.g., at least from 1 and up to 15 carbon or 30 carbon long alkyl spacers.

In one embodiment, the detectable label blocker is also the modifier group. A non-limiting example of such a modifier group is gold. Gold nanoparticles have been shown to quench fluorophores, e.g., described in Ghosh et al. Chemical Physics Letters, 2004, 395:366-372; Dulkeith et al. Nano Lett., 2005, 5:585-589; Mayilo et al. Nano Lett., 2009, 9:4558-4563; Dulkeith et al. Physical Review Letters, 2002, 89: 203002; Fan et al. PNAS, 2003, 100:6297-6301. These references are incorporated herein by reference in their entirety.

The main function of the modifier group is to add bulk to the oligonucleotide of the MB and in doing so adds bulk to the ds nucleic acid formed when a plurality of MBs are hybridized to a defined sequence that is representative of an A, U, T, C, or G nucleotide in a single-stranded nucleic acid to form the ds nucleic acid. The added bulk on the ds nucleic acid serves to (1) impede the ds nucleic acid from passing through a pore with a diameter opening of larger than 2.2 nm; (2) facilitate the use of a larger pore size nanopore for nanopore unzipping-dependent nucleic acid sequencing, and (3) aids in the unzipping of the plurality of MBs that are hybridized on a single stranded nucleic acid during nanopore unzipping-dependent nucleic acid sequencing. The unzipping is a sequential process. Shown in FIG. 9 is a ds nucleic acid undergoing the unzipping process as one strand translocates through the nanopore 120. The single-stranded nucleic acid 109 that translocates through the nanopore 120 having a pore width of D1 (101) is the define sequence that is representative of an A, U, T, C, or G nucleotide in the nucleic acid to be sequenced. The nucleic acid to be sequenced has been converted to the single-stranded 109 representative defined sequence for use in this nanopore unzipping DNA sequencing method. The ds nucleic acid comprises a single stranded sequence 109 and a plurality of MBs 111 complementarily hybridized thereon. Each MB comprises an oligonucleotide 117 with terminal fluorophores 105 and fluorophores quenchers 107, and a modifier group 103. The MBs shown in FIG. 9 have separate and distinct blocker and modifier group. As shown in FIG. 9, the width of the ds nucleic acid without the bulky modifier group is D2 (113). When D1 is greater than D2, a ds nucleic acid without a bulky modifier group can translocate through the nanopore of D1 width. The presence of a modifier group 103 increases the width of the ds nucleic acid with the bulky modifier group to D3 (115) which is greater that D1 (101). At the entrance to the nanopore 120, the MB 111 with the modifier group is “knocked” off from the single stranded nucleic acid 109 because the affinity between the MB 111 and the single stranded nucleic acid 109 is weaker that the affinity of the modifier group 103 to the MB 111.

The complementary hybridization of the MB 111 to the single-stranded nucleic acid 109 is by way of weak, non-covalent hydrogen bonds between the nucleobases on the MB and single-stranded nucleic acid. In some embodiments, the modifier group 103 is covalently linked to the MB 111. Since covalent bonds are stronger than hydrogen bonds, as the ds nucleic acid attempts to translocate the nanopore while in an electric field, the weaker hydrogen bonds breaks and the MB 111 are released from the ds nucleic acid. In other embodiments, the modifier group 103 is non-covalently linked to the MB 111, but this non-covalent linkage is stronger than hydrogen bonds. Non-covalent linkages that are be stronger that hydrogen bonds are ionic interactions and hydrophobic interactions. A non-limiting example of such non-covalent linkage is that of the avidin-biotin linkage that is well known in the art. The dissociation constant of avidin is measured to be Kd≈10⁻¹⁵ M, making it one of the strongest known non-covalent bonds. In one embodiment, the binding affinity between the hybridized single stranded nucleic acid and MBs is less than the binding affinity of the modifier group and the oligonucleotide of the MB, whereby the bond between the single stranded nucleic acid and MBs but not the bond between the modifier group and oligonucleotide of the MB becomes broken as the ds nucleic acid attempts to pass through the opening of the nanopore under the influence of an electric potential. In one embodiment, the hydrogen bonds between the hybridized single stranded nucleic acid and MBs are weaker than the ionic and/or hydrophobic interactions between the modifier group and the oligonucleotide of the MB.

In one embodiment, the modifier group is covalently linked to the oligonucleotide of the MB. In another embodiment, the modifier group is non-covalently linked to the oligonucleotide of the MB.

In one embodiment, the modifier group is selected from but is not limited to the group consisting of nanoscale particles, protein molecules, organometallic particles, metallic particles and semi conductor particles. The following are non-limiting examples of the types of modifier group contemplated herein. It is contemplated that any molecule that can add bulk to the MB when linked the MB and yet does not interfere with complementary base pairing can be used as the modifier group.

Nanoscale particles: any particle size under 1000 nm, e.g. TiO₂, gold, silver or latex beads, fullerenes (buckyballs), liposomes, silica-gold nanoshells and quantum dots. A vast variety of nanoparticles are commercially available, e.g., DYNABEADS from INVITROGEN, MAGNESPHERE form PROMEGA, and magnetic Beads from BIOCLONE. Conjugation of polystyrene latex nanobeads to DNA is described by Huang, et al., in Analytical Biochemistry 1996, 237:115-122 which is incorporated herein by reference in its entirety.

Protein molecules: DNA binding proteins, e.g., Zn finger proteins and histones; tat peptides; nuclear localization signal (NLS) peptide; streptavidin, avidin and various modified forms of avidin, e.g., neutravidin. DNA binding proteins naturally binds to DNA. In one embodiment, protein particles size ranges from 1-20 nm can be used. Other protein particles size ranges from 4-20 nm can be covalently linked to proteins through amide bond formation which are described in Taylor, J. R. et al., Analytical Chemistry 2000, 72: 1979-1986; Pagratis, N. Nucl. Acids Res. 1996, 24:3645-3646; Niemeyer, C. et al., Nucl. Acids Res. 1999, 27:4553-4561; Stahl, S. et al., Nucleic Acids Research 1988, 16:3025-3038; Sun, H. et al., Biosensors and Bioelectronics 2009, 24:1405-1410. These references are incorporated herein by reference in their entirety.

Organometallic particles: Ferrocene (0.5 nm) which can be conjugated by dimethoxytrityl nucleoside phosphoramidite coupling which is described by Ihara, T et al., in Nucl. Acids Res. 1996, 24:4273-4280; and Navarro, A.-E. et al., Bioorganic & Medicinal Chemistry Letters 2004, 14:2439-2441. These references are incorporated herein by reference in their entirety.

Metallic particles: Gold and silver coated gold (sized can range from 1.4-100 nm) and silver (25-30 nm). These can be conjugated to the MB oligonucleotide via cyclic disulfide, disulfide, thiol (sulfhydryls), and amine functional groups and also by biotin. These methods are detailly described in Mirkin, C. A. et al., Nature 1996, 382:607-609; Alivisatos, A. et al., Nature 1996, 382:609-611; Mucic, R. C et al., J. Amer. Chem. Soc. 1998, 120:2674-12675; Taton, T. A. et al., Science 2000, 289:1757-1760; Taton, T. A. et al., J. Amer. Chem. Soc. 2001, 123:5164-5165; Segond von Banchet, G., and Heppelman, B.: J. Histochem. Cytochem., 43, 821 (1995)); Letsinger, R. L et al., Bioconjugate Chemistry 2000, 11:289-291; Tokareva, I. and Hutter, E. J. Amer. Chem. Soc. 2004, 126:15784-15789; Lee, J.-S. et al., Nano Letters 2007, 7:2112-2115; Sun, H. et al., Biosensors and Bioelectronics 2009, 24:1405-1410. These references are incorporated herein by reference in their entirety.

Semi-conductor particles: Quantum dots and ZnS. A variety of semi-conductor type nanoparticles are commerically available, e.g., through INVITROGEN™. In one embodiment, semi-conductor particles having the size ranges of 15-20 nm can be used. These particles can be linked to the MB oligonucleotides via biotin, metal-thiol interactions, glycosidic bonding, electrostatic interactions or cysteine-capping the particle. The methods are described by Wu, S.-M. et al., Chem. Phys. Chem. 2006, 7:1062-1067; Xiao, Y. and Barker, P. E. Nucl. Acids Res. 2004, 32: e28; Yu, W. W. et al., Biochemical and Biophysical Research Communications 2006, 348:781-786; Artemyev, M. et al., J. Amer. Chem. Soc. 2004, 126:10594-10597; Li, Y. et al., Spectrochimica Acta Part A: Molecular and Biomolecular Spectroscopy 2004, 60: 1719-1724. These references are incorporated herein by reference in their entirety.

In one embodiment, the modifier group is located at the 5′ end or the 3′ end of the oligonucleotide of the MB. In another embodiment, the modifier group is located within 2-7 nucleotides from either the 3′ or 5′ end of the oligonucleotide of the MB. The modifier group can be located at the second nucleotide, at the third nucleotide, at the fourth nucleotide, at the fifth nucleotide, at the sixth nucleotide, or at the seventh nucleotide from either the 3′ or 5′ end of the oligonucleotide of the MB. In one embodiment, the modifier group is linked to the backbone of the oligonucleotide of the MB. The basic structure and components of a nucleic acid are known in the art. Nucleic acids are polymers composed of backbones and nucleobases, wherein the backbone comprises alternating sugar and phosphates or morpholinos. In another embodiment, the modifier group is linked to the nucleobases of the oligonucleotide of the MB. In some embodiments, the modifier group is linked to the oligonucleotide of the MB by a carbon linker. In some embodiments, the carbon linker has 1-30 carbons (alkyl) residues.

In one embodiment, the modifier group increases the width of a ds nucleic acid at the point of attachment of the modifier group to the oligonucleotide (D3) to greater than 2.0 nanometers (nm), wherein the ds nucleic acid is formed by hybridization of the MBs to the defined sequence that is representative of A, U, T, C, or G. In one embodiment, the modifier group increases the width D3 greater than 2.2 nm. In further embodiments, the modifier group increases the width D3 greater than 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.9, 9.0, 9.1, 9.2, 9.3, 9.4, 9.5, 9.6, 9.7, 9.8, 9.9, or 10 nm.

In one embodiment, the width (D3) of the ds nucleic acid at the point of attachment of the modifier group to the oligonucleotide of the MB is about 3-7 nm. In one embodiment, the width D3 is about 3-7 nm. In one embodiment, the width of the ds nucleic acid at the point of attachment of the modifier group to the single stranded nucleic acid can be further increased by a side-linker, e.g., C20, C15, C12, C9, C8, C6, C5, C4, C3 and C2 linkers.

In one embodiment, the modifier group on the oligonucleotide of the MB is 3-5 nm. In one embodiment, the modifier group ranges from 0.5 nm to 1000 nm. In one embodiment, the modifier group ranges from 90-944 nm. In one embodiment, the modifier group ranges from 4-20 nm. In one embodiment, the modifier group ranges from 1.4-100 nm. In one embodiment, the modifier group ranges from 25-30 nm. In one embodiment, the modifier group ranges from 15-20 nm. In one embodiment, the modifier group ranges from 15-30 nm. In one embodiment, the modifier group ranges from 150-300 nm. In one embodiment, the modifier group ranges from 9-50 nm. In one embodiment, the modifier group ranges from 10-100 nm. In other embodiments, the modifier group ranges from 3-1000 nm, 3-944 nm, 3-30 nm, 3-100 nm, 3-25 nm, 3-50 nm, 3-300 nm, 3-90 nm, 3-15 nm, 3-9 nm and 3-4 nm, including all the numbers to the second decimal place between 3 and 1000 nm.

In one embodiment, the modifier group facilitates the unzipping of the ds nucleic acid when the ds nucleic acid is subjected to nanopore sequencing.

In one embodiment of the methods described herein, the nanopore size permits the single stranded nucleic acid to be sequenced to pass through the pore, but not the ds nucleic acid to pass through the pore, wherein the ds nucleic acid is formed by the hybridization of the MBs described herein to the single stranded nucleic acid or a defined sequence that is representative of A, C, T, G or U.

In one embodiment of the methods described herein, the opening of the nanopore is larger than 2 nm but less than 1000 nm. In one embodiment, the opening of the nanopore is larger than 2 nm but less than the width of the ds nucleic acid at the point of attachment of the modifier group to the oligonucleotide of the MB.

In one embodiment of the methods described herein, the pore (D1) has an opening diameter of from about 3 nm to about 6 nm. In a further embodiment of the methods described herein, the pore has an opening diameter of from about 3 nm to up to 75% the width of the modifier group linked to the oligonucleotide of the MB. In certain embodiments of the methods described herein, the pore has a diameter from about 2.2 nm to 10 nm, from about 2.2 nm to 75 nm, or from about 2.2 nm to 100 nm, In further embodiments, the pore (D1) has a diameter of, for example, about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.9, 9.0, 9.1, 9.2, 9.3, 9.4, 9.5, 9.6, 9.7, 9.8, 9.9, or 10 nm in diameter.

In one embodiment of the methods described herein, the width (D3) of the ds nucleic acid at the point of attachment of the modifier group to the oligonucleotide of the MB is greater than 2 nm. In another embodiment of the methods described herein, the width (D3) of the ds nucleic acid at the point of attachment of the modifier group to the oligonucleotide of the MB is greater than 2.2 nm. In further embodiments of the methods described herein, the width (D3) of the ds nucleic acid at the point of attachment of the modifier group to the oligonucleotide of the MB is greater than 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.9, 9.0, 9.1, 9.2, 9.3, 9.4, 9.5, 9.6, 9.7, 9.8, 9.9, or 10 nm in diameter, wherein D3 is always greater than D1.

In one embodiment of the methods described herein, the width (D3) of the ds nucleic acid at the point of attachment of the modifier group to the oligonucleotide of the MB is about 3-5 nm. In one embodiment of the methods described herein, the width (D3) of the ds nucleic acid at the point of attachment of the modifier group to the oligonucleotide of the MB is about 3-6 nm. In other embodiments, D3 is about 3-7 nm, 3-8 nm, 3-9 nm, 3-10 nm, 3-12 nm, 3-15 nm, 3-17 nm or 3-20 nm.

In one embodiment of the methods described herein, D3 is greater than 2 nm. In another embodiment of the methods described herein, D3 is greater than 2.2 nm. In one embodiment, D3 is about 3-7 nm.

In one embodiment of the methods described herein, D1 is greater than 2 nm. In another embodiment of the methods described herein, D1 is greater than 2.2 nm. In one embodiment, D1 is about 3-6 nm.

In one embodiment of the methods described herein, the width (D3) of the ds nucleic acid at the point of attachment of the modifier group to the polymer is greater than the width of the opening (D1) of the nanopore, whereby as the ds nucleic acid attempts to pass through the opening under the influence of an electric potential, the modifier group blocks the MB on the ds nucleic acid from entering the opening and the MB unzips from the ds nucleic acid.

In one embodiment of the methods described herein, D3 is greater D1. In one embodiment, D1 is up to 75% of the width of D3.

In one embodiment of the methods described herein, the binding affinity between the hybridized single stranded nucleic acid and MBs is less than the binding affinity of the modifier group and the oligonucleotide of the MB, whereby the bond between the single stranded nucleic acid and MBs but not the bond between the modifier group and the oligonucleotide of the MB becomes broken as the ds nucleic acid attempts to pass through the opening of the nanopore under the influence of an electric potential. In one embodiment, the bond between the single stranded nucleic acid and MBs is a non-covalent hydrogen bond. In one embodiment, the bond between the modifier group and the oligonucleotide of the MB is a covalent bond. In one embodiment, the bond between the single stranded nucleic acid and MBs is a non-covalent hydrogen bond and the bond between the modifier group and the oligonucleotide of the MB is a a non-covalent bond such as ionic and hydrophobic interactions.

In one embodiment of the methods described herein, as the ds nucleic acid attempts to pass through the opening under the influence of an electric potential, the modifier group blocks the MB oligonucleotide on the ds nucleic acid from entering the opening, the non-covalent hydrogen bonds between the single stranded nucleic acid and MB oligonucleotides become broken. The MB oligonucleotides one by one sequentially and temporally separate and released from the single stranded nucleic acid at the entrance of the nanopore, wherein the single stranded nucleic acid enters the nanopore while the separated MBs do not.

In one embodiment of the methods described herein, the nucleic acid to be sequenced is a DNA or an RNA.

In one embodiment of the methods described herein, a single pore is employed. In another embodiment, multiple pores are employed.

The synthesis of MBs and methods of conjugation of an extraneous group to an oligonucleotide are known to one skilled in the art. Molecular beacons with the desired functional group can be synthesized using standard oligonucleotide synthesis techniques or purchased (e.g., from Integrated DNA Technologies). The skilled artisan will recognize that many additional molecular beacon sequences are commercially available and additional molecular beacon sequences can be designed for use in the methods of the present invention. A detailed discussion of the criteria for designing effective molecular beacon nucleotide sequences can be found on the World Wide Web at molecular-beacons organization and in Marras et al. (2003) “Genotyping single nucleotide polymorphisms with molecular beacons.” (In Kwok, P. Y. (ed.), Single nucleotide polymorphisms: methods and protocols. The Humana Press Inc., Totowa, N.J., Vol. 212, pp. 111-128); and Vet et al. (2004) “Design and optimization of molecular beacon real-time polymerase chain reaction assays.” (In Herdewijn, P. (ed.), Oligonucleotide synthesis: Methods and Applications. Humana Press, Totowa, N.J., Vol. 288, pp. 273-290), the contents of which are incorporated herein by reference in their entirety. Molecular beacons can also be designed using dedicated software, such as called “Beacon Designer”, which is available from Premier Biosoft International (Palo Alto, Calif.), the contents of which is incorporated herein by reference in its entirety.

Many modified nucleosides, nucleotides and various bases suitable for incorporation into nucleosides are commercially available from a variety of manufacturers, including the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), INVITROGEN™, San Diego, Calif., and Applied Biosystems (Foster City, Calif.), as well as many other commercial sources known to one of skill Methods of attaching bases to sugar moieties to form nucleosides are known. See, e.g., Lukevics and Zablocka (1991), Nucleoside Synthesis: Organosilicon Methods Ellis Horwood Limited Chichester, West Sussex, England and the references therein. Methods of phosphorylating nucleosides to form nucleotides and of incorporating nucleotides into oligonucleotides are also known. See, e.g., Agrawal (ed) (1993) Protocols for Oligonucleotides and Analogues, Synthesis and Properties, Methods in Molecular Biology volume 20, Humana Press, Towota, N.J., and the references therein. In addition, custom designed MBs are also commercially available, e.g., GENE TOOL LLC for Morpholinos; BIO-SYNTHESIS Inc. for PNA and chimeric PNA; and EXIQON for LNAs.

The modified nucleosides, nucleotides and various bases provide suitable linker for linking the detectable labels, detectable label blockers and the modifier group described herein. Linkers can be placed at the 3′ terminus, 5′ terminus or internally of the MB oligonucleotide. One skilled in the art would be able to select the appropriate linker and incorporate them during the synthesis of MBs. Non-limiting examples of amino linkers are 2′-Deoxyadenosine-8-C6 amino linker, 2′-Deoxycytidine-5-C6 amino linker, 2′-Deoxycytidine-5-C6 amino linker, 2′-Deoxyguanosine-8-C6 amino linker, 3′ C3 amino linker, 3′ C6 amino linker, 3′ C7 amino linker, 5′ C12 amino linker, 5′ C6 amino linker, C7 internal amino linker, thymidine-5-C2 and C6 amino linker, thymidine-5-C6 amino linker. Thiol linkers can be used to form either reversible disulfide bonds or stable thiol ether linkages with maleimides. Non-limiting examples of thiol linkers are 3′ C3 disulfide linker 3′ C6-disulfide linker and 5′ C6 disulfide linker. Other linkers include but are not limited to aldehyde linker for the 3′, aldehyde linker for the 5′ end, biotinylated-dT, carboxy-dT, and DADE linkers. Modified nucleosides, nucleotides and various bases for conjugation of extraneous group are commercially available, e.g., from TriLINK BIOTECHNOLOGIES.

In some embodiments, the detectable labels, the detectable label blocker and modifier groups are conjugated to the MB oligonucleotides by covalent linkage through spacers, preferably linear alkyl spacers. The chemical constituents of suitable spacers will be appreciated by persons skilled in the art. The length of a carbon-chain spacer can vary considerably, at least from 1 to 30 carbons.

In some embodiments, the MB oligonucleotide has extraneous group(s) linked to it. For example, groups can be linked to various positions on the nucleoside sugar ring or on the purine or pyrimidine rings which may stabilize the duplex by electrostatic interactions with the negatively charged phosphate backbone, or through hydrogen bonding interactions in the major and minor groves. For example, adenosine and guanosine nucleotides are optionally substituted at the N2 position with an imidazolyl propyl group, increasing duplex stability. Universal base analogues such as 3-nitropyrrole and 5-nitroindole are optionally included in oligonucleotide probes to improve duplex stability through base stacking interactions.

In certain embodiments, linking of the detectable labels, detectable label blockers and the modifier group occur by way of available primary amines (—NH₂) or secondary amines, carboxyls (—COOH), sulfhydryls/thiol (—SH), primary or secondary hydroxyl groups, and carbonyls (—CHO) functional groups on the Mb oligonucleotide and the label/blocker or modifier groups. One skilled in the art would recognize the available functional groups described herein or would de able to design and synthesize MB oligonucleotide or label/blocker or modifier group with desired function group for the purpose of conjugation. For example, in the instance where the peptide contains no available reactive thiol-group for chemical cross-linking, several methods are available for introducing thiol-groups into proteins and peptides, including but not limited to the reduction of intrinsic disulfides, as well as the conversion of amine or carboxylic acid groups to thiol group. Such methods are known to one skilled in the art and there are many commercial kits for that purpose, such as from Molecular Probes division of INVITROGEN™ Inc. and Pierce Biotechnology. In one embodiment, conjugation can takes place between protein's carboxyl group and amine groups on the amino linker on the MB oligonucleotide. The amino linker can be located at the 3′, 5′ or internal of the MB oligonucleotide.

Conjugation of several molecules using chemical cross-linking agents is well known in the art. Cross-linking reagents are commercially available or can be easily synthesized. One skilled in the art would be able to select the appropriate cross-linking agent based on the functional groups, e.g. disulfide bonds between cysteine amino acid residues in proteins, available for conjugation. Examples of cross-linking agents which should not be construed as limiting are glutaraldehyde, bis(imido ester), bis(succinimidyl esters), diisocyanates and diacid chlorides. Extensive data on chemical crosslinking agents can be found at INVITROGEN's Molecular Probe under section 5.2.

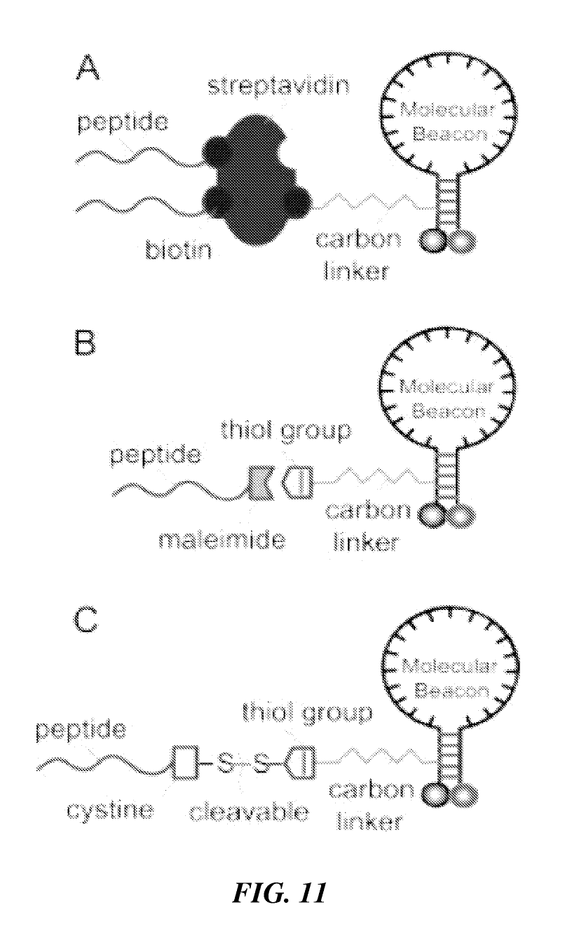

FIGS. 11A-C are examples of three different conjugation strategies for linking a peptide to molecular beacons. The conjugation strategies are applicable to any modifier group selected. FIG. 11A shows a streptavidin-biotin linkage in which a molecular beacon is modified by introducing a biotin-dT to the quencher arm of the stem through a carbon-12 spacer. The biotin-modified peptides are linked to the modified molecular beacon through a streptavidin molecule, which has four biotin-binding sites. The selected biotin-dT can have a spacer of varying length, for zero carbon up to 18 carbons.

FIG. 11B shows a thiol-maleimide linkage in which the quencher arm of the molecular beacon stem is modified by adding a thiol group which can react with a maleimide group placed to the C terminus of the peptide to form a direct, stable linkage. FIG. 11C shows a cleavable disulfide bridge in which the peptide is modified by adding a cysteine residue at the C terminus which forms a disulfide bridge with the thiol-modified molecular beacon. Thiol-dT is the most common method of adding a thiol group to an oligonucleotide. Thiol-dT can have a spacer of varying length, for zero carbon up to 18 carbons.

In one embodiment, the modifier group is linked to the detectable label arm of the MB oligonucleotide. In one embodiment, the modifier group is linked to the fluorophore arm of the MB oligonucleotide. In one embodiment, the modifier group is linked to the detectable label blocker arm of the MB oligonucleotide. In one embodiment, the modifier group is linked to the fluorophore quencher arm of the MB oligonucleotide.

In one embodiment, the signal emitted by the detectable group is fluorescence. Methods of detecting and measuring fluorescence are known to one skilled in the art, e.g. described in U.S. Pat. No. 6,191,852 and U.S. Patent Application Publication No. 20090056949. These references are incorporated herein by reference in their entirety.

Nanopore devices comprising synthetic or natural nanopores are known in the art and described herein. See, for example, Heng, J. B. et al., Biophysical Journal 2006, 90, 1098-1106; Fologea, D. et al., Nano Letters 2005 5(10), 1905-1909; Heng, J. B. et al., Nano Letters 2005 5(10), 1883-1888; Fologea, D. et al., Nano Letters 2005 5(9), 1734-1737; Bokhari, S. H. and Sauer, J. R., Bioinformatics 2005 21(7), 889-896; Mathe, J. et al., Biophysical Journal 2004 87, 3205-3212; Aksimentiev, A. et al., Biophysical Journal 2004 87, 2086-2097; Wang, H. et al., PNAS 2004 101(37), 13472-13477; Sauer-Budge, A. F. et al., Physical Review Letters 2003 90(23), 238101-1-238101-4; Vercoutere, W. A. et al., Nucleic Acids Research 2003 31(4), 1311-1318; Meller, A. et al., Electrophoresis 2002 23, 2583-2591. Nanopores and methods employing them are disclosed in U.S. Pat. Nos. 7,005,264 B2 and 6,617,113, U.S. Pat. Application Publication Nos. 2009/0029477 and 20090298072, and in Soni and Meller, Clin. Chem. 2007, 53:11. These references are incorporated herein by reference in their entirety.

The present invention can be defined in any of the following alphabetized paragraphs:
[A] A library of molecular beacons (MB) for nanopore unzipping-dependent sequencing of nucleic acids, the library comprising a plurity of MBs wherein each MB comprises an oligoucleotide that comprises (1) a detectable label; (2) a detectable label blocker; and (3) a modifier group; wherein the MB is capable of sequence-specific complementary hybridization to a defined sequence that is representative of an A, U, T, C, or G nucleotide in a single-stranded nucleic acid to form a double-stranded (ds) nucleic acid.
[B] The library of paragraph [A], wherein the oligonucleotide comprises 4-60 nucleotides.
[C] The library of paragraph [A] or [B], wherein the oligonucleotide of the MB comprises a nucleic acid selected from a group consisting of deoxyribonucleic acid (DNA), ribonucleic acid (RNA), peptide nucleic acid (PNA), locked nucleic acid (LNA) and phosphorodiamidate morpholino oligo (PMO or Morpholino).
[D] The library of any of paragraphs [A]-[C], wherein the detectable label is attached on one end of the oligonucleotide and is on the same end for all oligonucleotides in the library, wherein the detectable label emits a signal that can be detected and/or measured when the detectable label is not inhibited by the blocker.
[E] The library of any of paragraphs [A]-[D], wherein the MB is not attached to a solid phase carrier.
[F] The library of any of paragraphs [A]-[E], wherein the detectable label, detectable label blocker and the modifier group on the oligonucleotide do not interfere with sequence-specific complementary hybridization of the MB with the define sequence that is representative of an A, U, T, C, or G nucleotide in a single-stranded nucleic acid.
[G] The library of any of paragraphs [A]-[F], wherein the detectable group's signal is detected optically.
[H] The library of any of paragraphs [A]-[G], wherein the detectable group is a fluorophore and the signal is fluorescence.
[I] The library of any of claims [A]-[H], wherein the detectable label blocker is a quencher of the fluorophore.
[J] The library of any of paragraphs [A]-[I], wherein the detectable label blocker is also the modifier group.
[K] The library of any of paragraphs [A]-[J], wherein the modifier group is located at the 5′ end or the 3′ end of the oligonucleotide.
[L] The library of any of paragraphs [A]-[K], wherein the modifier group increases the width of the ds nucleic acid at the point of attachment of the modifier group to the oligonucleotide to greater than 2.0 nanometers (nm), wherein the ds nucleic acid is formed by hybridization of the MBs to the defined sequence that is representative of A, U, T, C, or G.
[M] The library of paragraph [L], wherein the width of the ds nucleic acid at the point of attachment of the modifier group to the oligonucleotide is about 3-7 nm.
[N] The library of any of claims [A]-[M] wherein the modifier group is selected from the group consisting of nanoscale particles, protein molecules, organometallic particles, metallic particles, and semi conductor particles.
[O] The library of any of paragraphs [A]-[N], wherein the modifier group is 3-5 nm.
[P] The library of any of paragraphs [A]-[O], wherein the modifier group facilitates the unzipping of the ds nucleic acid when the ds nucleic acid is subjected to nanopore sequencing.
[Q] The library of any of paragraphs [A]-[P], wherein there are two or more species of MBs, wherein each species of MB has a distinct detectable label.
[R] A method of unzipping a double-stranded (ds) nucleic acid for nanopore unzipping-dependent sequencing of nucleic acids, the method comprising
   a. hybridizing the library of molecular beacons (MBs) of claims [A]-[Q] to a single stranded nucleic acid to be sequenced, thereby forming a double stranded (ds) nucleic acid with a width of D3, which is formed by the presence of the modifier group, wherein the single stranded nucleic acid to be sequenced is a polymer comprising defined sequences representative of A, U, T, C or G;
   b. contacting the ds nucleic formed in step a) with an opening of a nanopore with a width of D1, wherein D3 is greater than D1; and
c. applying an electric potential across the nanopore to unzip the hybridized molecular beacons from the single stranded nucleic acid to be sequenced.
[S] The method of paragraph [R], wherein the nanopore size permits the single stranded nucleic acid to be sequenced to pass through the pore, but not the ds nucleic acid to pass through the pore.
[T] The method of paragraph [R] or [S], wherein D1 is greater than 2 nm.
[U] The method of any of paragraphs [R]-[T], wherein D1 is 3-6 nm.
[V] The method of any of paragraphs [R]-[U], wherein D3 is greater than 2 nm.
[W] The method of any of paragraphs [R]-[V], D3 is about 3-7 nm.
[X] The method of any of paragraphs [R]-[W], wherein the binding affinity between the hybridized single stranded nucleic acid and MBs is less than the binding affinity of the modifier group and the oligonucleotide of the MB, whereby the bond between the single stranded nucleic acid and MBs but not the bond between the modifier group and oligonucleotide of the MB becomes broken as the ds nucleic acid attempts to pass through the opening of the nanopore under the influence of an electric potential.
[Y] The method of any of paragraphs [R]-[X], wherein the nucleic acid to be sequenced is a DNA or RNA.
[Z] A method for determining the nucleotide sequence of a nucleic acid comprising the steps of:
   a. hybridizing the library of molecular beacons (MBs) of claims [A]-[Q] to a single stranded nucleic acid to be sequenced, thereby forming a double stranded (ds) nucleic acid with a width of D3, which is formed by the presence of the modifier group, wherein the single stranded nucleic acid to be sequenced is a polymer comprising defined sequences representative of A, U, T, C or G;
   b. contacting the ds nucleic formed in step a) with an opening of a nanopore with a width of D1, wherein D3 is greater than D1;
   c. applying an electric potential across the nanopore to unzip the hybridized MBs from the single stranded nucleic acid to be sequenced; and
   d. detecting a signal emitted by a detectable label from each MB as the MB separate from the ds nucleic acid as it occurs at the pore.
[AA] The method of paragraph [Z] further comprising decoding the sequence of detected signals to the nucleotide base sequence of the nucleic acid.
[BB] The method of paragraph [Z] or [AA], wherein the nanopore size permits the single stranded nucleic acid to be sequenced to pass through the pore, but not the ds nucleic acid to pass through the pore.
[CC] The method of any of paragraphs [Z]-[BB], wherein D1 is greater than 2 nm.
[DD] The method of any of paragraphs [Z]-[CC], wherein D1 is about 3-6 nm.
[EE] The method of any of paragraphs [Z]-[DD], wherein D3 is greater than 2 nm.
[FF] The method of any of paragraphs [Z]-[EE], wherein D3 is about 3-7 nm.
[GG] The method of any of paragraphs [Z]-[FF], wherein the binding affinity between the hybridized single stranded nucleic acid and MBs is less than the binding affinity of the modifier group and the oligonucleotide of the MB, whereby the bond between the single stranded nucleic acid and MBs but not the bond between the modifier group and oligonucleotide of the MB becomes broken as the ds nucleic acid attempts to pass through the opening of the nanopore under the influence of an electric potential.
[HH] The method of any of paragraphs [Z]-[GG], wherein the nucleic acid to be sequenced is a DNA or an RNA.

This invention is further illustrated by the following example which should not be construed as limiting. The contents of all references cited throughout this application, as well as the figures are incorporated herein by reference.

### Example

### Optical Recognition of Individual Nucleobases for Single-Molecule DNA Sequencing with Nanopore Arrays

### Introduction

High-throughput DNA sequencing technologies are profoundly impacting comparative genomics, biomedical research, and personalized medicine'. In particular, single-molecule DNA sequencing techniques minimize the amount of required DNA material, and therefore are considered to be prominent candidates for delivering low-cost and high-throughput sequencing, targeting a broad range of DNA read lengths¹⁻⁴. Solid-state nanopores are one class of single-molecule probing techniques that have extensive applications, including characterization of DNA structure and DNA-drug or DNA-protein interactions⁵⁻¹². Unlike other single-molecule techniques, detection with nanopores does not require immobilization of macromolecules onto a surface, thus simplifying sample preparation. Furthermore solid-state nanopores can be fabricated in high-density format, which will allow the development of massively parallel detection.

A nanopore is a nanometer-sized pore in an ultra-thin membrane that separates two chambers containing ionic solutions. An external electrical field applied across the membrane creates an ionic current and a local electrical potential gradient near the pore, which draws in and threads biopolymers through the pore in a single file manner^{6,13}. As a biopolymer enters the pore, it displaces a fraction of the electrolytes, giving rise to a change in the pore conductivity, which can be measured directly using an electrometer. A number of nanopore based DNA sequencing methods have recently been proposed¹⁴ and highlight two major challenges¹⁵: 1) The ability to discriminate among individual nucleotides (nt). The system must be capable of differentiating among the four bases at the single-molecule level. 2) The method must enable parallel readout. As a single nanopore can probe only a single molecule at a time, a strategy for manufacturing an array of nanopores and simultaneously monitoring them is needed. Recently it was demonstrated that individual nucleotides can be identified using a modified sa-hemolysin protein pore after cleavage of the DNA bases with an exonuclease¹⁶. The kinetics of enzymatic activity, however, remains the rate-limiting step for readout. Furthermore, the throughput of this method, as well as other single-molecule methods that involve enzymes at the readout stage, is restricted by the processivity of the enzyme, which varies greatly from molecule to molecule. To date, parallel readout through any nanopore-based method has not yet been demonstrated.

The inventors present a novel nanopore-based method for high-throughput base recognition that obviates the need for enzymes during the readout stage and provides a straightforward method for multi-pore detection. Biochemical preparation of the target DNA molecules converts each base into a form that can be read directly using an unmodified solid-state nanopore. Readout speed and length are therefore not enzyme limited. While previous publications utilized electrical signals to probe biomolecules in nanopores, here the inventors use optical sensing to detect DNA sequence. The inventors have developed a custom Total Internal Reflection (TIR) method, which permits high spatiotemporal resolution wide-field optical detection of individual DNA molecules translocating through a nanopore¹⁷. Here the inventors use this system to achieve simultaneous optical detection from multiple nanopores. Thus the inventors demonstrate the proof of principle for all of the key components of a nanopore-based single-molecule sequencing method.

### Methods

Electrical measurements: Nanochips were fabricated in-house, starting from a double-sided polished silicon wafer coated with 30 nm thick, low-stress SiN using LPCVD. SiN windows (30×30 μm²) were created using standard procedures. Nanopores (3-5 nm in diameter) were fabricated using a focused electron beam, as previously described²⁸. The drilled nanochips were cleaned and assembled on a custom-designed CTFE cell incorporating a glass coverslip bottom (see ref¹⁷ for details) under controlled humidity and temperature. Nanopores were hydrated with the addition of degassed and filtered 1M KCl electrolyte to the cis chamber and 1M KCl with 8.6M urea to the trans chamber to facilitate Total Internal Reflection (TIR) imaging through the trans chamber, as explained below. All electrolytes were adjusted to pH 8.5 using 10 mM Tris-HCl. Ag/AgCl electrodes were immersed into each chamber of the cell and connected to an Axon 200B headstage used to apply a fixed voltage (300 mV for all experiments) across the membrane and to measure the ionic current when needed. The fluid cell was placed inside a custom Faraday box to reduce noise pick-up, which was mounted on a modified inverted microscope. Nanopore current was filtered using a 50 kHz low pass Butterworth filter and sampled using a DAQ board at 250 kHz/16 bit (PCI-6 154, National Instruments, TX). The signals were acquired using a custom LabView program as previously described⁹.

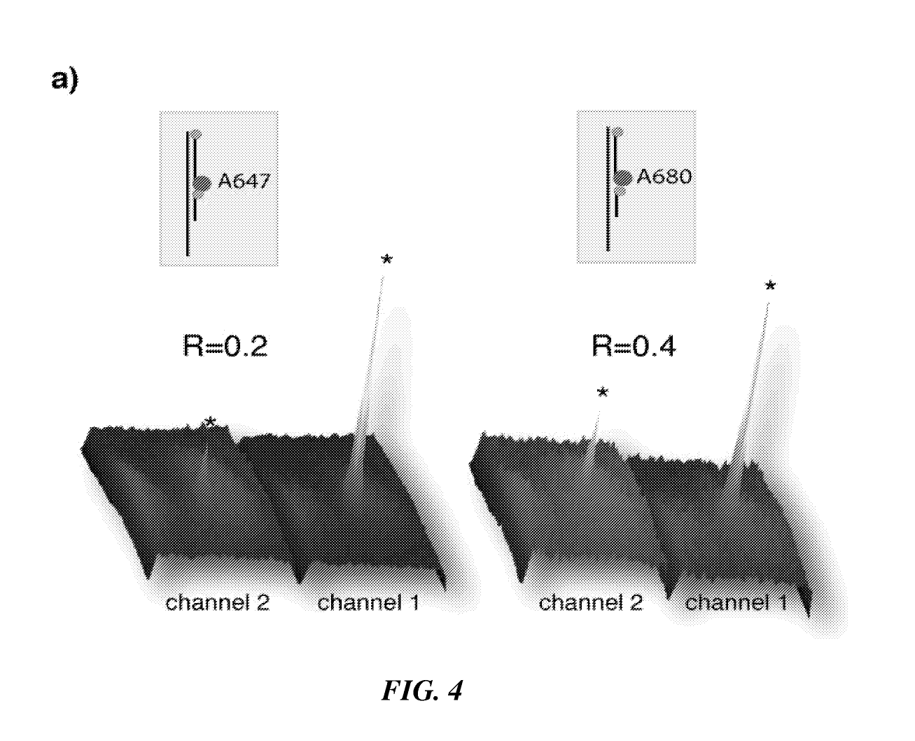

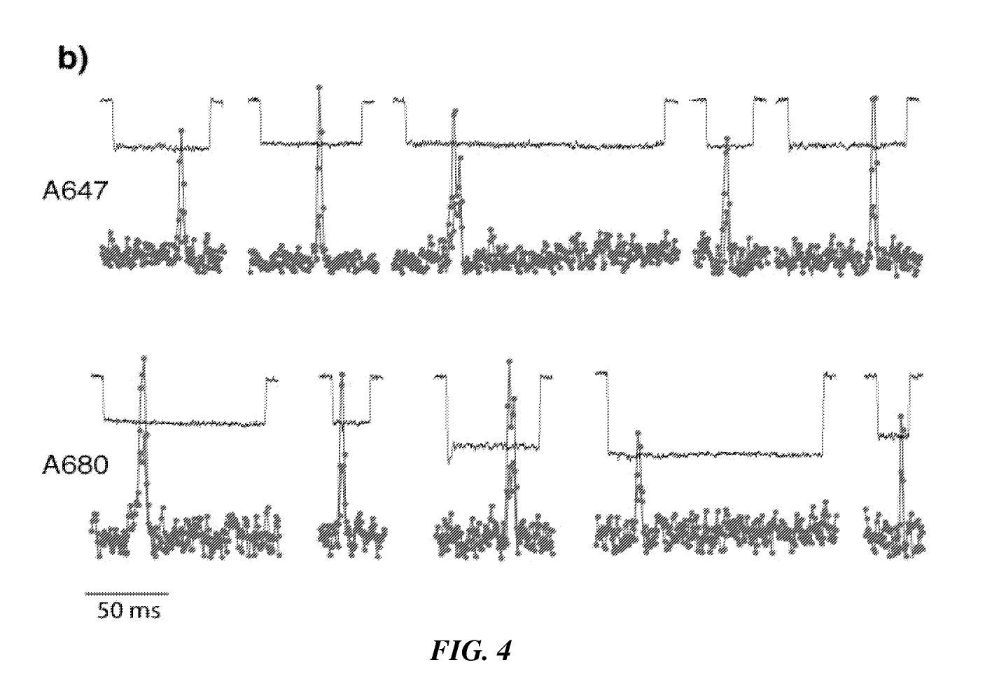

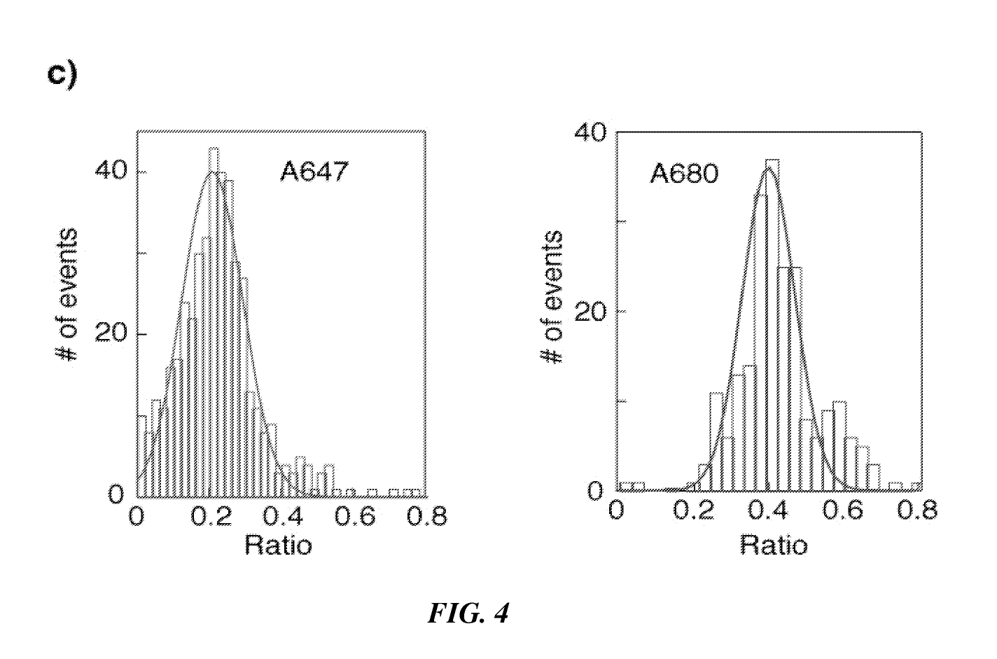

Electrical/optical detection and signal synchronization: To achieve high-speed single molecule detection of individual fluorophores near the suspended SiN membrane, a custom TIR imaging was developed, which greatly reduces the fluorescence background¹⁷. The index of refraction of the trans chamber solution was adjusted, such that TIR could be created at the SiN membrane, preventing light from progressing into the cis chamber thus reducing additional background. The cell was mounted on a high NA objective (Olympus 60×/1.45), and TIR was optimized by focusing the incident laser beam 640 nm laser (20 mW, iFlex2000, Point-Source UK) to an off-axis point at its back focal plane, thereby controlling the angle of incidence. Fluorescence emission was split into two separate optical paths using a Semrock (FF685-Di01) dichroic mirror and the two images were projected side by side onto an EM-CCD camera (Andor, iXon DU-860). The EM-CCD worked at maximum gain and 1 ms integration time. Synchronization between the electrical and optical signals was achieved by connecting the camera ‘fire’ pulse to a counter board (PCI-6602, National Instruments, TX), which shared the same sampling clock and start trigger as the main DAQ board. The combined data stream included unique time stamps at the beginning of each CCD frame, which were synched with the ion current sampling. Two separate criteria were used for classifying each event. First, the ion current must abruptly drop below a user defined threshold level, and remain at that level for at least 100 μs before returning to the origina state. Second, the corresponding CCD frames during the event dwell-time (time where signal stays below the threshold), must show increase in the photon count, only at the region of the pore. Two-color intensity analysis was performed by reading the intensity at a 3×3 pixel area centered at the pore position (see for example FIG. 4a). The raw intensity data in the two channels was used to calculate the ratio R=Ch2/Ch1, used to discriminate between the two bits. Discrimination was done automatically in a custom LabView code, using the calibration data (FIG. 4c). Data analysis was performed using IGOR Pro (Wavemetrics), and fits were created to optimize chi-square.

### Preparing Avidin-Biotinylated Molecular Beacons

As the avidin/strepavidin molecules contain 4 binding sites, it was imperative that only a single molecular beacon bind to one avidin protein molecule. As such, it was found that pre-incubation for 30 min with a molar ratio of 3:1 free biotin to avidin/strepavidin in Tris-EDTA buffer served as a well suited priming step. After which, the biotinylated DNA beacons was added to the solution such that the ratio of beacons to avidin/strepavidin was 5:1. This ensured that only 1 beacon bound to one avidin protein molecule.

### Results

The approach comprises two steps (FIG. 1a): First, each of the four nucleotides (A, C, G and T) in the target DNA, i.e., the DNA to be sequenced, is converted to a predefined sequence of oligonucleotides, which is hybridized with a molecular beacon that carries a specific fluorophore. For two-color readout (i.e., two types of fluorophores), the four sequences are combinations of two predefined unique sequences bit ‘0’ and bit ‘1’, such that an A would be ‘1, 1’, a G would be ‘1,0’, a T would be ‘0,1’ and finally a C would be ‘0,0’ (FIG. 1a, left panel). Two types of molecular beacons carrying two types of fluorophores hybridize specifically to the ‘0’ and ‘1’ sequences. Second, the converted DNA and hybridized molecular beacons are electrophoretically threaded through a solid-state pore, where the beacons are sequentially stripped off. Each time a beacon is stripped off, a new fluorophore is unquenched, giving rise to a burst of photons, recorded at the location of the pore (FIG. 1a, right panel). The sequence of two-color photon bursts at each pore location (the colors are converted different shades of grey in FIG. 1) is the binary code of the target DNA sequence. The inventors approach addresses the two challenges facing nanopore sequencing: 1) circumvent the need for detecting individual bases and facilitate an enzyme-free readout; and 2) wide-field imaging and spatially fixed pores enable straightforward adaptation to simultaneous detection of multiple pores with a electron multiplying charge coupled device (EM-CCD) camera (schematically illustrated in FIG. 1b).

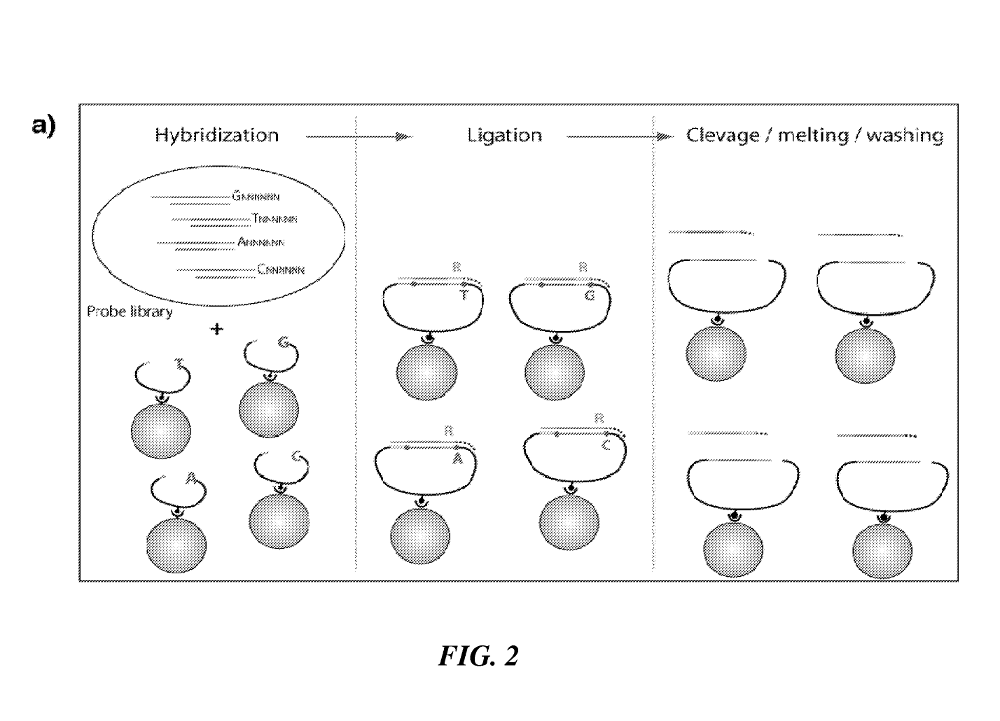

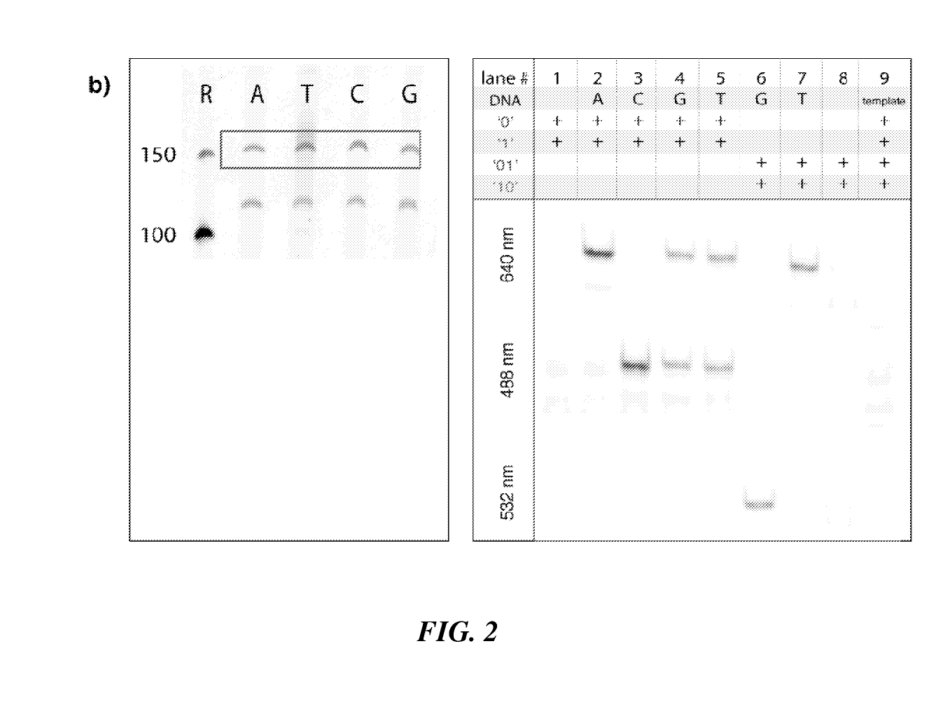

FIG. 2 illustrates the conversion of target DNA, as a process that is named Circular DNA Conversion (CDC) because a circular DNA molecule is formed during each cycle of the conversion. FIG. 2a displays schematically the three steps of CDC, and FIG. 2b displays the results of a single conversion cycle. For proof of principle, four single stranded DNA (ssDNA) templates were synthesized, all four templates were 100-nt long and they differ only in their 5′-end nucleotide. These templates contain a biotin moiety for immobilization onto streptavidin-coated magnetic beads. In the initial step, these templates are hybridized to a library of DNA molecules (called probes), each with a double-stranded center portion and two single-stranded overhangs. The double-stranded portion contains the predefined oligonucleotide code that matches the 5′-end nucleotide of the template molecule. Only those probes whose 3′ overhangs perfectly complement the 5′ end of a template can hybridize with the template. The 5′ overhang of the probe hybridizes with the 3′ end of the same template to form a circular molecule. In the second step of the conversion, a T4 DNA ligase is used to ligate both ends of the probe with the template (the two locations of ligation are indicated by red dots in FIG. 2a). T4 DNA ligase has been used in other DNA sequencing methods due to its extremely high fidelity compared with other enzymes¹⁸. Finally, the double-stranded portion of the probe contains the recognition site of a type IIS restriction enzyme (labeled with an ‘R’) and positions it to cleave right after the 5′-end nucleotide of the template. After a brief thermally induced melting and subsequent washing, the newly formed ssDNA contains, at its 3′-end, the binary code followed by the 5′-end nucleotide of the original template. This process can be repeated as many times as needed, transferring nucleotides from the 5′-end of the template to the 3′-end, interdigitated with the corresponding codes. The conversion of different template molecules does not need to be synchronized, and unproductive hybridization will not lead to error, as long as no ligation and cleavage ensue.

### Circular DNA Conversion (CDC)

The purpose of the conversion process is to have each individual base, in a DNA template, be represented by longer predefined sequence. For proof of concept purpose, four DNA template molecules (100-mer each) were synthesized where each template only differs by the identity of the terminal 5′ base. These templates contain a biotin moiety for immobilization of the templates onto streptavidin coated magnetic beads (INVITROGEN DYNABEADS MYONE Streptavidin C1). This immobilization step enables the quick removal, and replacement, of buffer solutions during the differing stages of the conversion process, with minimal lost of DNA samples. Template molecules are first suspended with the beads in a buffer solution (2M NaCl, 2 mM EDTA, 20 mM Tris) for 10 minutes to allow immobilization to occur. This is followed by a wash step to remove the immobilization buffer solution. The coated beads are then resuspended in a solution containing a library of DNA molecules that are referred to herein as probes. Each probe is a sticky-ended, double stranded, molecule that contains the predefined oligonucleotide code for a specific base, as shown in FIG. 2a. Only those probes whose 3′ overhangs perfectly complement the 5′-end of a template can hybridize with the template. The library probes are designed to allow the 3′ end of the template molecules to hybridize to the 5′ overhang of the probes. The sample is then run through a slow-cool process to allow the library probes to hybridize to their complementary template molecule. This process is carried out at high salt (100 mM NaCl, 10 mM MgCl₂) to promote hybridization. At this stage in the process a circular molecule has been created. The sample is then washed with a 10 mM Tris buffer solution, to remove any excess library probes that have not hybridized to the immobilized template molecules. The sample is then re-suspended in a ligation buffer solution to allow the newly hybridized molecules to ligate together. The ligation buffer solution contains Quick T4 DNA Ligase (New England BioLabs) and a Quick Ligation Reaction buffer (New England BioLabs). Ligation is carried out at room temperature for 5 minutes. After this step another wash is carried out with 10 mM Tris buffer solution, to remove the ligase and ligation buffer solution. The penultimate step of the conversion process is to resuspend the newly circularized and immobilized molecules in a buffer solution containing BseG1 restriction enzyme and a FASTDIGEST buffer (both from Fermantes). This process re-linearizes the circularized molecule in such a way that the predefined code, plus the base that it represents, now reside at the 3′ end of the template molecule, and a new base now sits at the 5′ end, ready to go through the process of conversion. Once the sample has been suspended in this digestion buffer it is left for 15 minutes at 37° C. to allow digestion to take place.

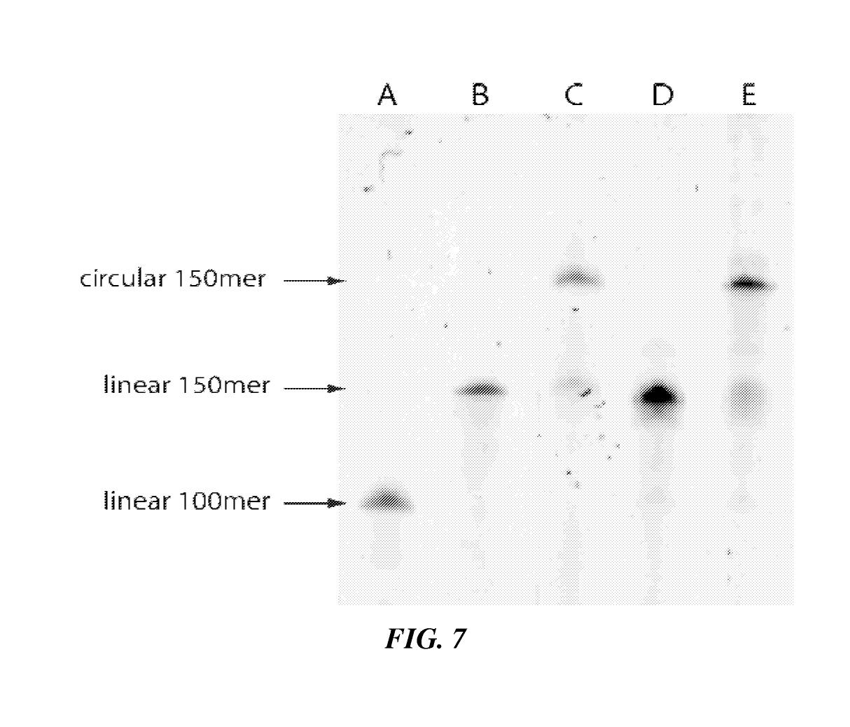

To analyze the molecules using either nanopore or gels, the converted DNA was removed from the beads. This is done by suspending the immobilized sample in a 95% formamide buffer and heating to 95° C. for 10 minutes. The sample is then run on a denaturing gel (FIG. 2b and FIG. 7) to verify the conversion. FIG. 7 displays a denaturing gel of some of the key stages of the process (here only C-terminal template is shown for clarity). This gel was stained using SYBR Green II, (INVITROGEN). The gel shows: A. The original DNA template molecule. B. A linear 150 mer ssDNA shown as a reference. C. A circular 150 mer DNA shown as reference. D. The converted product after linearization using BseG1. E. The converted circularized product before linearization. These display the extended length of the molecule after the hybridization, ligation and digestion steps.

### DNA Sequences Used for Proof of Principles of Circular DNA Conversion (CDC)

Below are the sequences for the molecular beacons used to verify the identity of the converted products described previous in the example. All the beacon sequences below were synthesized by Eurogentec NA San Diego:

A. 1 6-mer Complementary to the “1” bit. 5′-TAAGCGTACGTGCTTA-3′ (SEQ. ID. NO. 13).

This sequence has a 5′ amine modification and an ATTO647N (Atto-Tec) dye was conjugated at the 5′ end. For nanopore optical readout experiment, the same oligonucleotide (molecular beacon) was synthesized with a quencher (BHQ-2, Biosearch Technologies) at the 3′ end.

B. 16mer complementary to the “0” bit: 5′-CCTGATTCATGTCAGG-3′ (SEQ. ID. NO.14). This sequence has a 5′ amine modification and an ATTO488 (Atto-Tec) dye was conjugated at the 5′ end. For nanopore optical readout experiment, the same oligo was synthesized with a quencher (BHQ-2, Biosearch Technologies) at the 3′ end, an ATTO680 (Atto-Tec) dye was conjugated at the 5′ end.

C. 32mer complementary to the “01” sequence: 5′-CCTGATTCATGTCAGGTAAGCGTACGTGCTTA-3′ (SEQ. ID. NO. 15). This sequence has a 5′ amine modification and an ATTO647N (Atto-Ttec) dye was conjugated at the 5′ end.

D. 32mer complementary to the “10” sequence: 5′-TAAGCGTACGTGCTTACCTGATTCATGTCAGG-3′ (SEQ. ID. NO. 16). This sequence has a 5′ amine modification and a TM R (INVITROGEN™) dye was conjugated at the 5′ end.

The inventors extensively tested the feasibility of CDC by analyzing the reaction products after their removal from the magnetic beads. The left panel of FIG. 2b displays a denaturing gel (8 M urea) containing the product after one run of conversion. It was observed that >50% of each of the four different templates were extended by ˜50 nts (from 100 to ˜1 50 nts), indicating successful ligation of the template with a probe. To prove that the correct probe was used in each case, four types of oligonucleotides were synthesized, also known as molecular beacons, as follows: 1) a 16-mer complementary to the “1” bit, with a red fluorophore; 2) a 16-mer complementary to the “0” bit, with a blue fluorophore; 3) a 32-mer complementary to the “10” two-bit sequence, with a green fluorophore; and 4) a 32-mer complementary to “01”, with a red fluorophore. A mixture of the first two oligonucleotides was hybridized to each CDC product, and as a control, to all four initial templates. After gel separation, image analysis was carried out using a 3-color laser scanner and displayed in FIG. 2c. The colors were converted to grey scales in the Figures. Only one red band for the “A” product was observed, and only one blue band for the “C” product, coded as “11” and “00” respectively (lane 2 and 3) was observed. The other two products, “G” and “T” display both a red and a blue band, as they are coded by “10” and “01” respectively (lane 4 and 5). To distinguish between the converted “G” and “T”, they were hybridized with the aforementioned two 32-mer oligonucleotides. Only “G” di plays a band labeled with the green fluorophore, corresponding to the “10” code (lane 6) and only “T” displays a band labeled with the red fluorophore, corresponding to the “01” code (lane 7) Controls show that the templates themselves do not hybridize to any of the labeled molecular beacons, and that the labeled molecular beacons themselves do not show in the gel as they are too short compared with the ˜150 nt products (lanes 1, 8 and 9). These results conclusively show that a single CDC cycle produces pure products with the correct conversion codes.

The second step of the inventors approach uses a solid-state nanopore to strip hybridized molecular beacons off converted ssDNA. This requires the use of pores in the sub-2 nm range, because the cross-section diameter of double stranded DNA (dsDNA) is 2.2 nm¹⁹. The probability of DNA molecules' entry into such small pores is much smaller than their entry into larger pores^{9,13}, necessitating the use of a larger amount of DNA. Moreover, manufacturing small pores poses many technical challenges, as there is little tolerance for error, and the difficulty escalates for high-density nanopore arrays. It was found that covalently attaching a 3-5 nm sized “bulky” group (eg. a protein or a nanoparticle) to the molecular beacons effectively increases the molecular cross section of the complex to 5-7 nm, allowing the use of nanopores in the size range of 3-6 nm. This increases the capture rate of DNA molecules by 10 fold or more, and greatly facilitates the fabrication process of the nanopore arrays.

For proof of concept, an avidin (4.0×5.5×6.0 nm)²⁰ molecule was attached to a biotinylated molecular beacon containing a fluorophore-quencher pair (ATTO647N-BHQ2, abbreviated as “A647-BHQ”) Both this beacon and a similarly constructed molecular beacon, containing a quencher at one end and no fluorophore at the other end, were hybridized to a target ssDNA (‘1-bit’ sample). A similar complex was synthesized containing two beacon molecules (‘2-bit’ sample), as shown schematically in FIG. 3a.

### Bulk Fluorescence Studies

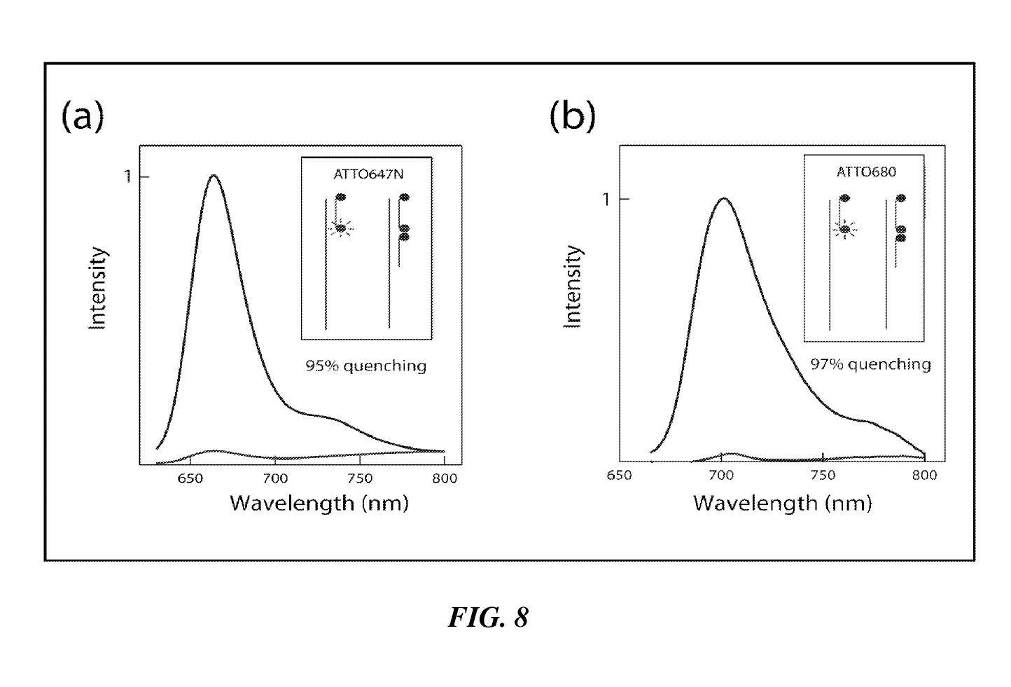

In order to test the efficiency of the quenching process of BHQ-2, bulk fluorescence experiments were carried out. For each fluorophores, two molecules were designed (see insets to FIGS. 8 (a) and (b)). One molecule consisted of a 16mer, containing a fluorescent dye at its 5′ end, hybridized to a 66 mer. The second molecule again contained the same 16mer plus a second 16mer which contained BHQ-2 quencher at its 3′ end. These two 1 6mers were hybridized to a 66mer. The two 16mer molecules were hybridized such that the fluorescent probe on the 5′ end of one was in close proximity to the BHQ-2 quencher on the 3′ end of the other. The two fluorophores used were ATTO647N (Atto-Tec) and ATTO680 (Atto-Tec). ATTO647N has a maximum absorption peak at 644 nm and an excitation peak at 669 nm, while ATTO680 has a maximum absorption peak at 680 nm and an excitation peak at 700 nm. For each molecule, we used a spectrofluorometer (JASCO FP-6500) to measure the fluorescence emissions of the complexes. Initially the emission spectrums of the molecules were measured with the unquenched fluorophores (top traces in (a) and (b) of FIG. 8). Then the emissions spectrum of the molecules with a quencher-fluorophore pair (bottom traces in (a) and (b) of FIG. 8) were measured. Each experiment contained ˜100 nM of hybridized sample. These experiments determined that there is 95-97% quenching occurring for these bulk molecules, as indicated in FIG. 8.

Therefore, the bulk studies demonstrated that, when in its hybridized state, the A647 fluorophore on the molecular beacon is quenched ˜95% by the neighboring BHQ quencher. Given this extremely high quenching efficiency, fluorescence bursts can be detected at the single-molecule level only if strand separation occurs as that is when the fluorophores is not next to an adjacent quencher in the hybridized double-stranded state.

Nanopore experiments for both the 1-bit and 2-bit samples were carried out using a 640 nm laser and imaged at 1,000 frames per second using an EM-CCD camera. FIG. 3a displays typical unzipping events for the two samples, with one beacon per complex in the 1-bit sample, and two beacons per complex in the 2-bit sample. Electrical signals are shown in black, and optical signals, measured synchronously with the electrical signals at the pore position¹⁷, in light grey or dark grey traces. An abrupt decrease in electrical current signifies the entry of the molecule to the pore, and when the pore is cleared the electrical signal returns to the open-pore upper state¹⁹. The optical signals clearly show either one or two photon bursts for the vast majority of unzipping events in the 1-bit and 2-bit samples, respectively. This is expected since the fluorophores are quenched before reaching the pore and are self-quenched again immediately after the beacons are unzipped from the template²¹. Summation of the optical intensity during each unzipping event as defined by the electrical signal, yielded Poisson distributions for the two samples (solid lines in FIG. 3b), with mean value 1.30±0.06 for the 1-bit sample, and double value (2.65±0.08) for the 2-bit sample (n>600 events in each case, errors represent std). This proves that regardless of a model used to define a photon burst, on average a single unzipping event occurred for each complex in the 1-bit sample and two unzipping events occurred for the 2-bit samples. Moreover, with the use of an intensity threshold analysis (chosen at the average intensity+2 std) it was observed that nearly 90% of the collected events in the 1 bit sample contained a single fluorescent burst, while in the 2 bit sample, ˜80% of the collected events displayed 2 such bursts (FIG. 3c). This data demonstrates that it is possible to optically discriminate between 1 bit and 2 bit samples, in individual unzipping events performed using a 3-5 nm pore.

To distinguish between all four nucleotides, the current system was extend from a 1 color to a 2 color coding scheme using two high quantum yield fluorophores, A647 (ATTO647N) and A680 (ATTO680), excited simultaneously by the same 640 nm laser. The optical emission signal was split into channels 1 and 2 using a dichroic mirror and imaged side-by-side on the same EM-CCD camera. As the emission spectra of the two fluorophores overlap, a fraction of the A647 emission “leaks” into channel 2, and a fraction of A680 “leaks” to channel 1. Two calibration measurements were performed using 1-bit complexes labeled with A647 or A680 fluorophores (FIG. 4a). Clearly seen is a single distinct peak in each channel, corresponding to the location of the nanopore, after accumulation of >500 unzipping events in each case. The ratio of the fluorescent intensities in Channel 2 vs. Channel 1 (R) is 0.2 for the A647 sample, and 0.4 for the A680 sample.

Representative events (out of >500) for each for the two samples, and the corresponding distributions of R, are depicted in FIGS. 4b and 4c, respectively. A single prominent fluorescent peak was observed during each translocation event (electrical traces shown in black), with intensity>3 fold larger than the baseline fluorescence fluctuations. Tallying up all detected events led to R=0.20±0.06 and 0.40±0.05 (mean±std) for A647 and A680, respectively, in complete agreement with the ratios for accumulated fluorescence (for all events) shown in FIG. 4a. R follows a Gaussian distribution, given by the solid line fits in FIG. 4c. These control measurements show that R can used to determine the identity of individual fluorophores.

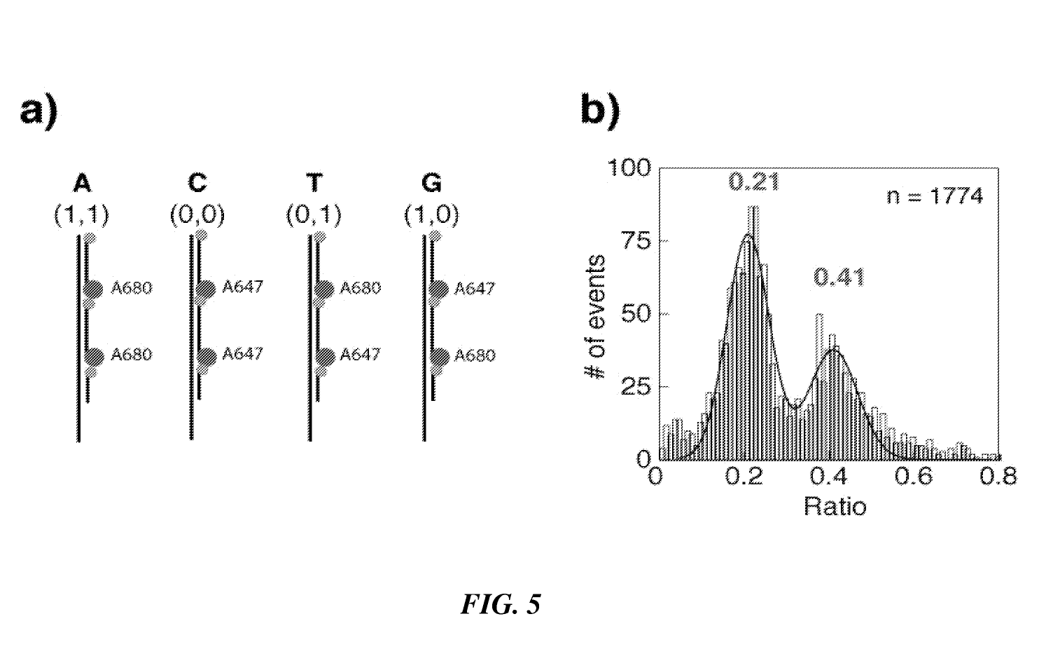

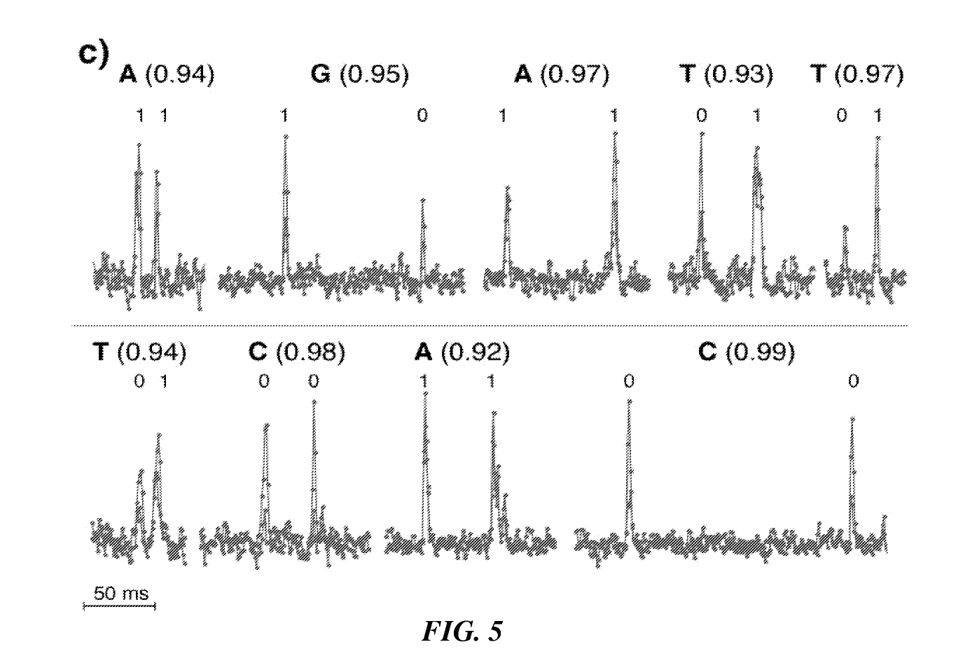

Using the calibration distributions given in FIG. 4c, the ability to identify the products from the CDC containing the four 2-bit combinations, namely 11 (A), 00 (C), 01 (T), and 10 (G), where “0” and “1” correspond to the A647 and A680 beacons, respectively was tested. Analysis of >2000 unzipping events revealed a bimodal distribution of R, with two modes at 0.21±0.05 and 0.41±0.06 (FIG. 5b), in complete agreement with the calibration measurements (FIG. 4c). All photon bursts with R<0.30 was classified as “0”, and those with R>030 was classified as “1” (0.30 is the local minimum of the distribution in FIG. 5b). The distribution of R was also used to compute the probability of misclassification. This further provides a statistical means to calibrate the two channels for optimal discrimination between the two fluorophores. FIG. 5c presents representative 2-color fluorescence intensity events depicting the single molecule identification of all 4 DNA bases.

The robustness of the two-color identification is attributed primarily to the excellent signal-to-noise ratio of the photon bursts and the separation between the fluorophore intensity ratios for the two channels. A computer algorithm was developed to perform automatic peak identification in fluorescence signals. The algorithm filters out random noise (e.g. false spikes) in the fluorescence signals and identifies the bit sequence using the calibration distributions (FIG. 4c), and then performs base calling. The algorithm outputs two certainty scores, one for bit calling and the other one for base calling. Typical results are shown in FIG. 5c. The certainty value for each base extracted automatically from the raw intensity data (range between 0 and 1) is displayed in parenthesis.

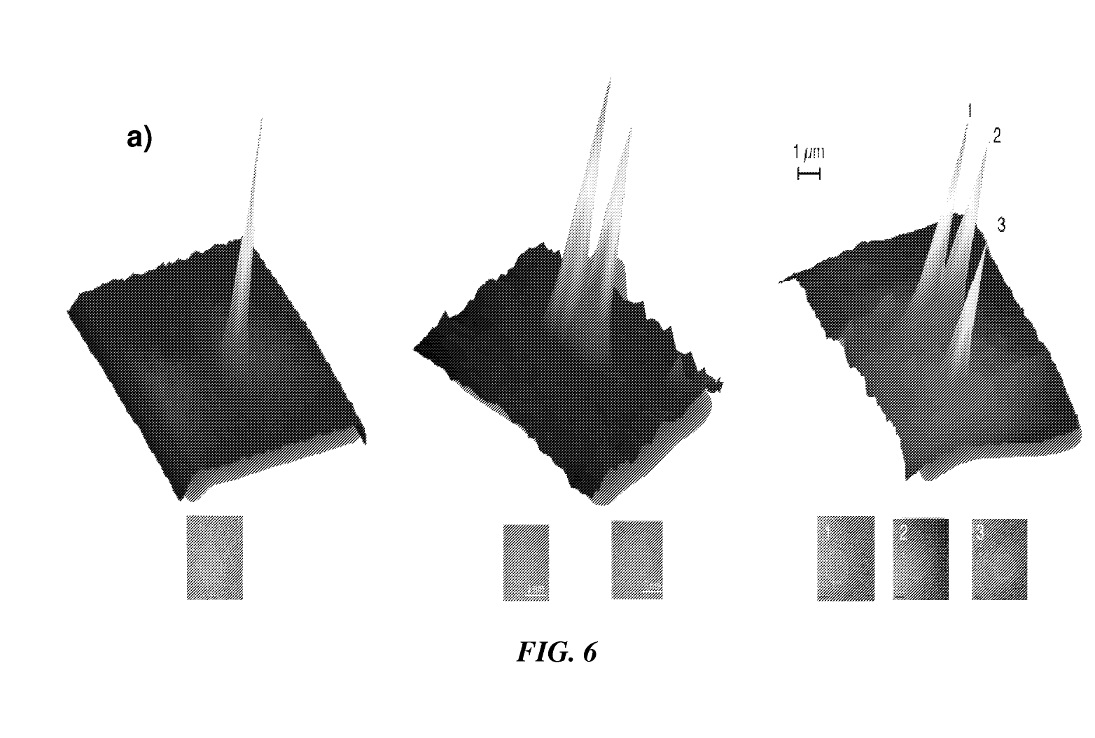

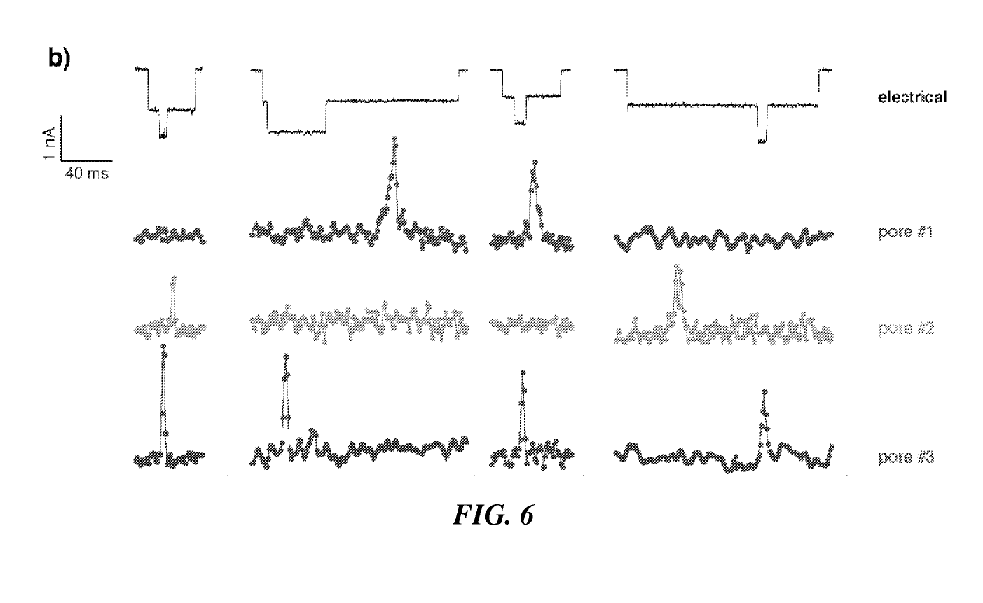

One of the major advantages of the current wide-field optical-based detection scheme lies in the simplicity with which multiple pores can be probed in parallel, ultimately enabling high-throughput readout. As a proof of concept for parallel readout, multiple 3-5 nm sized nanopores on the same SiN membrane were fabricated, separated by several microns. In FIG. 6a display the accumulated fluorescence intensity images, obtained in three separate experiments, using membranes containing one, two or three nanopores. Like the single pore experiments, fluorescent bursts from all pores in the membrane were recorded. Accumulating photon counts from several thousand unzipping events in each experiment resulted in surface maps of photon intensity at each pixel (FIG. 6a). As reflected in the figure, the number of peaks detected equals the number of pores fabricated in each membrane. The distance between the two peaks for the two-pore membrane was 1.8 μm, and the distances between the three peaks for the three-pore membrane were 1.8 μm and 7.7 μm, in complete agreement with the distances between the pores measured during the fabrication process. This data provides direct evidence for the feasibility of a wide-field optical detection scheme.

FIG. 6b demonstrated the ability of the system to probe photon bursts simultaneously from multiple nanopores in a single membrane. Four representative traces show the electrical current (black) and the optical signal using 1-bit sample probed from the three nanopores (green, red and blue markers, respectively). The entrance and unzipping of each molecule, at each pore, is a stochastic process. Under the conditions used in this experiment, out of >3,000 unzipping events, ˜50 involved molecules entering through two pores at the same time. The electrical current trace, which is accumulated from all pores, displays two distinct blockade levels, indicating the total number of occupied pores at a particular moment, without information on which pores are occupied. The optical traces on the other hand reveal occupied pores unambiguously. This will ultimately eliminate the need for electrical current measurements when the method extends to larger arrays, and rely solely on optical measurements, simplifying instrumentation requirements.

### DISCUSSION AND CONCLUSION

Single-molecule DNA sequencing methods have already begun to transform genetic research, setting a higher bar for cost and throughput^{3,22,23}. It is anticipated that as the cost of sequencing is further decreased, human genome re-sequencing will become a widespread and affordable medical diagnostic tooll. Here it has been demonstrated the feasibility of a new single-molecule DNA sequencing concept that has the potential to be at low cost and ultra high throughput. In its simplest form, a binary code (2 bits per base) was used to represent a DNA sequence, which is coupled with two fluorophores and read by an optical detection system. At its current stage, the current system can read 50-250 bases per second per nanopore, which compares favorably with other single-molecule approaches^{2,3}. It is anticipated that a straightforward adaptation for 4-color and the use of optimized reagent will allow the system to achieve >500 bases per second per nanopore. Most importantly, the feasibility of multi-pore readout was demonstrated, the first time for nanopore based methods. Optical detection from nanopore arrays scales efficiently with the number of pores, unlike enzymatic methods that rely on statistical occupancy.

The inventors approach contains a preparatory step to convert the target DNA into longer DNA molecules that can be directly probed with a standard solid-state nanopore. Despite the added time and complexity, this step brings the following advantages: 1) Unlike other sequencing platforms²⁴, this approach does not require a PCR-based amplification step, which can be error prone². 2) The readout stage does not use any enzymes such as polymerase, ligase or exonuclease, hence the readout length, speed, and fidelity are not enzyme limited 3) The readout speed can be easily regulated for individual sequencing reactions, by adjusting physical parameters such as the voltage across the nanopore, or the ionic strengths in the two chambers. An enzyme-dependent method would require bioengineering of the involved enzymes. 4) The converted DNA can be designed to possess little secondary structure, which can greatly facilitate sequencing of highly structured and/or repetitive regions in the genome, circumventing the need for strong denaturants in the readout stage. 5) The readout system uses standard solid-state nanopore arrays in the size range 3-6 nm, which can be manufactured en masse.

The inventors' results herein demonstrate the first all solid-state DNA sequence readout and the incorporation of a bulky group allows the use of 3-6 nm pores. These results strongly indicate the feasibility of using solid-state nanopores for DNA sequencing. Recently, a number of publications have demonstrated the fabrication of similar scale arrays in solid-state materials^{25,26}.

### REFERENCES

1. Shendure, J., et al., Advanced sequencing technologies: Methods and goals. Nature Reviews Genetics 5 (5), 335-344 (2004).
2. Harris, T. D. et al., Single-molecule DNA sequencing of a viral genome. Science 320 (5872), 106-109 (2008).
3. Eid, J. et al., Real-time DNA sequencing from single polymerase molecules. Science 323 (5910), 133-138 (2009).
4. Fuller, C. W. et al., The challenges of sequencing by synthesis. Nature Biotechnology 27 (11), 1013-1023 (2009).
5. Li, J. et al., Ion-beam sculpting at nanometre length scales. Nature 412, 166-169 (2001).
6. Deamer, D. W. & Branton, D., Characterization of nucleic acids by nanopore analysis. Accounts of Chemical Research 35 (10), 817-825 (2002).
7. Healy, K., Nanopore-based single-molecule DNA analysis. Nanomedicine 2 (4), 459-481 (2007).
8. Dekker, C., Solid-state nanopores. Nature Nanotechnology 2 (4), 209-215 (2007).
9. Wanunu, M., et al., DNA Translocation Governed by Interactions with Solid-State Nanopores. Biophysical Journal 95 (10), 4716-4725 (2008).
10. Wanunu, M., Sutin, J., & Meller, A., DNA profiling using solid-state nanopores: Detection of DNA-binding molecules. Nano Letters 9 (10), 3498-3502 (2009).
11. Singer, A. et al., Nanopore-based sequence-specific detection of duplex DNA for genomic profiling. Nano Letters 10 (2), 738-742 (2010).
12. Liu, H. et al., Translocation of Single-Stranded DNA Through Single-Walled Carbon Nanotubes. Science 327 (5961), 64-67 (2010).
13. Wanunu, M., et al., Electrostatic Focusing of Unlabeled DNA into Nanoscale Pores using a Salt Gradient. Nature Nanotechnology 5, 160-165 (2009).
14. Vercoutere, W. & Akeson, M., Biosensors for DNA sequence detection. Curr. Opin. Chem. Biol. 6 (6), 8 16-822 (2002).
15. Branton, D. et al., The potential and challenges of nanopore sequencing. Nature Biotechnology 26 (10), 1146-1153 (2008).
16. Clarke, J. et al., Continuous base identification for single-molecule nanopore DNA sequencing. Nature Nanotechnology 4 (4), 265-270 (2009).
17. Soni, V. G. et al., Synchronous optical and electrical detection of bio-molecules traversing through solid-state nanopores. Rev. Sci. Instru. 81 (1), 014301-014307 (2010).
18. Shendure, J. et al., Accurate multiplex polony sequencing of an evolved bacterial genome. Science 309 (5741), 1728-1732 (2005).
19. McNally, B., Wanunu, M., & Meller, A., Electromechanical unzipping of individual DNA molecules using synthetic sub-2 nm pores. Nano Letters 8 (10), 3418-3422 (2008).
20. Green, N. M. & Joynson, M. A., A preliminary crystallographic investigation of avidin. Biochem J 118 (1), 71-72 (1970).
21. Bonnet, G., Krichevsky, O., & Libchaber, A., Kinetics of conformational fluctuations in DNA hairpin-loops. Proc. Natl. Acad. Sci. USA 95 (15), 8602-8606 (1998).
22. Lipson, D. et al., Quantification of the yeast transcriptome by single-molecule sequencing. Nature Biotechnology 27 (7), 652-U105 (2009).
23. Pushkarev, D., Neff, N. F., & Quake, S. R., Single-molecule sequencing of an individual human genome. Nature Biotechnology 27 (9), 847-U101 (2009).
24. Li, Y. & Wang, J., Faster human genome sequencing (News and Views). Nature Biotechnology 27 (9), 820-821 (2009).
25. Tong, H. D. et al., Silicon nitride nanosieve membrane. Nano Letters 4 (2), 283-287 (2004).
26. Hopman, W. C. L. et al., Focused ion beam scan routine, dwell time and dose optimizations for submicrometre period planar photonic crystal components and stamps in silicon. Nanotechnology 18 (19), 195305-195311 (2007).
27. Pipper, J. et al., Catching bird flu in a droplet. Nature Medicine 13 (10), 1259-1263 (2007).
28. Kim, M. J., Wanunu, M., Bell, D. C., & Meller, A., Rapid fabrication of uniformly sized nanopores and nanopore arrays for parallel DNA analysis. Advanced Materials 18 (23), 3149-3153 (2006).
29. Soni G. V. and Meller A., Progress towards ultrafast DNA sequencing using solid-state nanopores. Clinical Chemistry 53, 11 (2007).
30. Meller A., et al., Ultra high-throughput opti-nanopore DNA readout platform. U.S. Patent Application No. US 2009/0029477.
31. Preben Lexon, Sequencing method using magnifying tags. U.S. Pat. No. 6,723,513.
32. Ju, Jingyue, Dna sequencing by nanopore using modified nucleotides. U.S. Patent Application US 2009/0298072

1. A library of molecular beacons for nanopore unzipping-dependent sequencing of nucleic acids, the library comprising a plurality of molecular beacons wherein each molecular beacon comprises an oligonucleotide that comprises
(1) a detectable label;
(2) a detectable label blocker; and
(3) a modifier group;
wherein the molecular beacon is capable of sequence-specific complementary hybridization to a defined sequence that is representative of an A, U, T, C, or G nucleotide in a single-stranded nucleic acid to form a double-stranded nucleic acid.

2. The library of claim 1, wherein the oligonucleotide comprises 4-60 nucleotides.

3. The library of claim 1, wherein the oligonucleotide of the molecular beacon comprises a nucleic acid selected from a group consisting of deoxyribonucleic acid (DNA), ribonucleic acid (RNA), peptide nucleic acid (PNA), locked nucleic acid (LNA) and phosphorodiamidate morpholino oligo (PMO or Morpholino).

4. The library of claim 1, wherein the detectable label is attached on one end of the oligonucleotide and is on the same end for all oligonucleotides in the library, wherein the detectable label emits a signal that can be detected and/or measured when the detectable label is not inhibited by the blocker.

5. The library of claim 1, wherein the molecular beacon is not attached to a solid phase carrier.

6. The library of claim 1, wherein the detectable label, detectable label blocker and the modifier group on the oligonucleotide do not interfere with sequence-specific complementary hybridization of the MB with the define sequence that is representative of an A, U, T, C, or G nucleotide in a single-stranded nucleic acid.

7. The library of claim 4, wherein the signal of the detectable label is detected optically.

8. The library of claim 4, wherein the detectable group is a fluorophore and the signal is fluorescence.

9. The library of claim 1, wherein the detectable label blocker is a quencher of the fluorophore.

10. The library of claim 1, wherein the detectable label blocker is also the modifier group.

11. The library of claim 1, wherein the modifier group is located at the 5′ end or the 3′ end of the oligonucleotide.

12. The library of claim 1, wherein the modifier group increases the width of the double-stranded nucleic acid at the point of attachment of the modifier group to the oligonucleotide to greater than 2.0 nanometers (nm), wherein the double-stranded nucleic acid is formed by hybridization of the molecular beacons to the defined sequence that is representative of A, U, T, C, or G.

13. The library of claim 12, wherein the width of the double-stranded nucleic acid at the point of attachment of the modifier group to the oligonucleotide is about 3-7 nm.

14. The library of claim 1, wherein the modifier group is selected from the group consisting of nanoscale particles, protein molecules, organometallic particles, metallic particles, and semiconductor particles.

15. The library of claim 1, wherein the modifier group is 3-5 nm.

16. The library of claim 1, wherein the modifier group facilitates unzipping of the double-stranded nucleic acid when the ds nucleic acid is subjected to nanopore sequencing.

17. The library of claim 1, wherein there are two or more species of molecular beacons, wherein each species of molecular beacon has a distinct detectable label.

18. A method of unzipping a double-stranded nucleic acid for nanopore unzipping-dependent sequencing of nucleic acids, the method comprising:
a. hybridizing the library of molecular beacons of claim 1 to a single stranded nucleic acid to be sequenced, thereby forming a double stranded nucleic acid with a width of D3, which is formed by the presence of the modifier group, wherein the single stranded nucleic acid to be sequenced is a polymer comprising defined sequences representative of A, U, T, C or G;
b. contacting the double stranded nucleic formed in step a) with an opening of a nanopore with a width of D1, wherein D3 is greater than D1; and
c. applying an electric potential across the nanopore to unzip the hybridized molecular beacons from the single stranded nucleic acid to be sequenced.

19. The method of claim 18, wherein the nanopore size permits the single stranded nucleic acid to be sequenced to pass through the pore, but not the double stranded nucleic acid to pass through the pore.

20. The method of claim 18, wherein D1 is greater than 2 nm.

21. The method of claim 20, wherein D1 is 3-6 nm.

22. The method of claim 18, wherein D3 is greater than 2 nm.

23. The method of claim 22, wherein D3 is about 3-7 nm.

24. The method of claim 18, wherein the binding affinity between the hybridized single stranded nucleic acid and molecular beacons is less than the binding affinity of the modifier group and the oligonucleotide of the molecular beacon, whereby the bond between the single stranded nucleic acid and molecular beacons but not the bond between the modifier group and oligonucleotide of the molecular beacon becomes broken as the double stranded nucleic acid attempts to pass through the opening of the nanopore under the influence of an electric potential.

25. The method of claim 18, wherein the nucleic acid to be sequenced is a DNA, or a RNA.

26. A method for determining the nucleotide sequence of a nucleic acid comprising:
a. hybridizing the library of molecular beacons of claim 1 to a single stranded nucleic acid to be sequenced, thereby forming a double stranded nucleic acid with a width of D3, which is formed by the presence of the modifier group, wherein the single stranded nucleic acid to be sequenced is a polymer comprising defined sequences representative of A, U, T, C or G;
b. contacting the double-stranded nucleic acid formed in step a) with an opening of a nanopore with a width of D1, wherein D3 is greater than D1;
c. applying an electric potential across the nanopore to unzip the hybridized molecular beacons from the single stranded nucleic acid to be sequenced; and
d. detecting a signal emitted by a detectable label from each molecular beacon MB as the molecular beacon separates from the double-stranded nucleic acid as it occurs at the pore.

27. The method of claim 26, further comprising decoding the sequence of detected signals to the nucleotide base sequence of the nucleic acid.

28. The method of claim 26, wherein the nanopore size permits the single stranded nucleic acid to be sequenced to pass through the pore, but not the double-stranded nucleic acid to pass through the pore.

29. The method of claim 26, wherein D1 is greater than 2 nm.

30. The method of claim 29, wherein D1 is about 3-6 nm.

31. The method of claim 26, wherein D3 is greater than 2 nm.

32. The method of claim 31, wherein D3 is about 3-7 nm.

33. The method of claim 26, wherein the binding affinity between the hybridized single stranded nucleic acid and molecular beacons is less than the binding affinity of the modifier group and the oligonucleotide of the molecular beacon, whereby the bond between the single stranded nucleic acid and molecular beacons but not the bond between the modifier group and oligonucleotide of the molecular beacon becomes broken as the double-stranded nucleic acid attempts to pass through the opening of the nanopore under the influence of an electric potential.

34. The method of claim 26, wherein the nucleic acid to be sequenced is a DNA or an RNA.


SEQUENCE LISTING (16 sequences)

SEQ ID NO: 1 - DNA, 18 bp, Artificial Sequence
  Description of Artificial Sequence Synthetic oligonucleotide
atttggaatttccgaggt

SEQ ID NO: 2 - DNA, 36 bp, Artificial Sequence
  Description of Artificial Sequence Synthetic oligonucleotide
gcgagctaggaaacaccaaagatgatatttgctcgc

SEQ ID NO: 3 - DNA, 10 bp, Artificial Sequence
  Description of Artificial Sequence Synthetic oligonucleotide
atttattagg

SEQ ID NO: 4 - DNA, 10 bp, Artificial Sequence
  Description of Artificial Sequence Synthetic oligonucleotide
cgggcggcaa

SEQ ID NO: 5 - DNA, 10 bp, Artificial Sequence
  Description of Artificial Sequence Synthetic oligonucleotide
cctttcctta

SEQ ID NO: 6 - DNA, 10 bp, Artificial Sequence
  Description of Artificial Sequence Synthetic oligonucleotide
agcgccgaac

SEQ ID NO: 7 - DNA, 50 bp, Artificial Sequence
  Description of Artificial Sequence Synthetic oligonucleotide
cgggcggcaaagcgccgaacagcgccgaaccctttccttaatttattagg

SEQ ID NO: 8 - DNA, 20 bp, Artificial Sequence
  Description of Artificial Sequence Synthetic oligonucleotide
atttattaggcgggcggcaa

SEQ ID NO: 9 - DNA, 20 bp, Artificial Sequence
  Description of Artificial Sequence Synthetic oligonucleotide
atttattaggatttattagg

SEQ ID NO: 10 - DNA, 20 bp, Artificial Sequence
  Description of Artificial Sequence Synthetic oligonucleotide
cgggcggcaaatttattagg

SEQ ID NO: 11 - DNA, 20 bp, Artificial Sequence
  Description of Artificial Sequence Synthetic oligonucleotide
cgggcggcaacgggcggcaa

SEQ ID NO: 12 - DNA, 140 bp, Artificial Sequence
  Description of Artificial Sequence Synthetic polynucleotide
cgggcggcaacgggcggcaaatttattaggcgggcggcaaatttattaggatttattaggcgggcggcaacgggcggcaacgggcggcaacgggcggcaacgggcggcaaatttattaggatttattaggcgggcggcaa

SEQ ID NO: 13 - DNA, 16 bp, Artificial Sequence
  Description of Artificial Sequence Synthetic oligonucleotide
taagcgtacgtgctta

SEQ ID NO: 14 - DNA, 16 bp, Artificial Sequence
  Description of Artificial Sequence Synthetic oligonucleotide
cctgattcatgtcagg

SEQ ID NO: 15 - DNA, 32 bp, Artificial Sequence
  Description of Artificial Sequence Synthetic oligonucleotide
cctgattcatgtcaggtaagcgtacgtgctta

SEQ ID NO: 16 - DNA, 32 bp, Artificial Sequence
  Description of Artificial Sequence Synthetic oligonucleotide
taagcgtacgtgcttacctgattcatgtcagg